SEQUENCE LISTING (21 sequences)

SEQ ID NO: 1 - DNA, 18 bp, Artificial Sequence
  Synthetic sequence
ggggtttctactggtggt

SEQ ID NO: 2 - DNA, 21 bp, Artificial Sequence
  Synthetic sequence
cccctckgsaaagccttcttc

SEQ ID NO: 3 - DNA, 18 bp, Artificial Sequence
  Synthetic sequence
tggtaygacatkawatgg

SEQ ID NO: 4 - DNA, 18 bp, Artificial Sequence
  Synthetic sequence
rcgsggcargaacatsgg

SEQ ID NO: 5 - DNA, 15 bp, Artificial Sequence
  Synthetic sequence
tgcgtggartgycac

SEQ ID NO: 6 - DNA, 17 bp, Artificial Sequence
  Synthetic sequence
agrtargakysggcaaa

SEQ ID NO: 7 - DNA, 21 bp, Artificial Sequence
  Synthetic sequence
taygtsgggcaggaraaactg

SEQ ID NO: 8 - DNA, 22 bp, Artificial Sequence
  Synthetic sequence
cgtagaagaagctggtgctgtt

SEQ ID NO: 9 - DNA, 20 bp, Artificial Sequence
  Synthetic sequence
taccacccsgarccgcgcgt

SEQ ID NO: 10 - DNA, 18 bp, Artificial Sequence
  Synthetic sequence
gccgccgtcrtgvaggaa

SEQ ID NO: 11 - DNA, 19 bp, Artificial Sequence
  Synthetic sequence
gacaarhwvtaytggtggt

SEQ ID NO: 12 - DNA, 18 bp, Artificial Sequence
  Synthetic sequence
  misc_feature (4)..(4): n is a, c, g, or t
  misc_feature (10)..(10): n is a, c, g, or t
  misc_feature (13)..(13): n is a, c, g, or t
  misc_feature (16)..(16): n is a, c, g, or t
gaanccccanacnccngc

SEQ ID NO: 13 - DNA, 20 bp, Artificial Sequence
  Synthetic sequence
cgytgttcmtcgacagccag

SEQ ID NO: 14 - DNA, 20 bp, Artificial Sequence
  Synthetic sequence
cgsaccttsttgccstygcg

SEQ ID NO: 15 - DNA, 19 bp, Artificial Sequence
  Synthetic sequence
tcaccaccgacggcaagac

SEQ ID NO: 16 - DNA, 20 bp, Artificial Sequence
  Synthetic sequence
acgatcatcagcatcttggc

SEQ ID NO: 17 - DNA, 1389 bp, Thauera sp. strain SND5
cgggggcttcggcctgccggcgagtggcgaacgggtgagtaatgcatcggaacgtgcccatgtcgtgggggataacgtatcgaaaggtacgctaataccgcatacgccctgagggggaaagcgggggattcttcggaacctcgcgcgattggagcggccgatgtcggattagctagtaggtgaggtaaaggctcacctaggcgacgatccgtagcgggtctgagaggatgatccgccacactgggactgagacacggcccagactcctacgggaggcagcagtggggaattttggacaatgggcgcaagcctgatccagccatgccgcgtgagtgaagaaggccttcgggttgtaaagctctttcggccgggaagaaatcgcgtgctctaacatagcgcgtggatgacggtaccggactaagaagcaccggctaaattacgtgccagcagccgcggtaatacgtagggtgcgagcgttaatcggaattactgggcgtaaagcgtgcgcaggcggttttgtaagacagatgtgaaatccccgggcttaacctgggaactgcgtttgtgactgcaaggctagagtacggcagaggggggtggaattcctggtgtagcagtgaaatgcgtagagatcaggaggaacaccgatggcgaaggcagccccctgggcctgtactgacgctcatgcacgaaagcgtggggagcaaacaggattagataccctggtagtccacgccctaaacgatgtcgactagtcgttcggagcagcaatgcactgagtgacgcagctaacgcgtgaagtcgaccgcctggggagtacggccgcaaggttaaaactcaaaggaattgacggggacccgcacaagcggtggatgatgtggattaattcgatgcaacgcgaaaaaccttacctacccttgacatgtctggaaccttgctgagaggcgagggtgccttcgggagccagaacacaggtgctgcatggctgtcgtcagctcgtgtcgtgagatgttgggttaagtcccgcaacgagcgcaacccttgtcactagttgccatcatttagttgggcactctagtgagactgccggtgacaaaccggaggaaggtggggatgacgtcaagtcctcatggcccttatgggtagggcttcacacgtcatacaatggtcggtacagagggttgccaagccgcgaggtggagccaatcccttaaagccgatcgtagtccggatcgtagtctgcaactcgactacgtgaagtcggaatcgctagtaatcgcagatcagcatgctgcggtgaatacgttcccgggtcttgtacacaccgcccgtcacaccatgggagtgggtttcaccagaagtaggtagcttaaccttcgggagggcgct

SEQ ID NO: 18 - DNA, 1542 bp, Thauera sp. strain SND5
tgaacttaagagtttgatcctggctcagattgaacgctggcggcatgctttacacatgcaagtcgaacggcagcgggggcttcggcctgccggcgagtggcgaacgggtgagtaatgcatcggaacgtgcccatgtcgtgggggataacgtatcgaaaggtacgctaataccgcatacgccctgagggggaaagcgggggattcttcggaacctcgcgcgattggagcggccgatgtcggattagctagtaggtgaggtaaaggctcacctaggcgacgatccgtagcgggtctgagaggatgatccgccacactgggactgagacacggcccagactcctacgggaggcagcagtggggaattttggacaatgggcgcaagcctgatccagccatgccgcgtgagtgaagaaggccttcgggttgtaaagctctttcggccgggaagaaatcgcgtgctctaacatagcgcgtggatgacggtaccggactaagaagcaccggctaactacgtgccagcagccgcggtaatacgtagggtgcgagcgttaatcggaattactgggcgtaaagcgtgcgcaggcggttttgtaagacagatgtgaaatccccgggcttaacctgggaactgcgtttgtgactgcaaggctagagtacggcagaggggggtggaattcctggtgtagcagtgaaatgcgtagagatcaggaggaacaccgatggcgaaggcagccccctgggcctgtactgacgctcatgcacgaaagcgtggggagcaaacaggattagataccctggtagtccacgccctaaacgatgtcgactagtcgttcggagcagcaatgcactgagtgacgcagctaacgcgtgaagtcgaccgcctggggagtacggccgcaaggttaaaactcaaaggaattgacggggacccgcacaagcggtggatgatgtggattaattcgatgcaacgcgaaaaaccttacctacccttgacatgtctggaaccttgctgagaggcgagggtgccttcgggagccagaacacaggtgctgcatggctgtcgtcagctcgtgtcgtgagatgttgggttaagtcccgcaacgagcgcaacccttgtcactagttgccatcatttagttgggcactctagtgagactgccggtgacaaaccggaggaaggtggggatgacgtcaagtcctcatggcccttatgggtagggcttcacacgtcatacaatggtcggtacagagggttgccaagccgcgaggtggagccaatcccttaaagccgatcgtagtccggatcgtagtctgcaactcgactacgtgaagtcggaatcgctagtaatcgcagatcagcatgctgcggtgaatacgttcccgggtcttgtacacaccgcccgtcacaccatgggagtgggtttcaccagaagtaggtagcttaaccttcgggagggcgcttaccacggtgagattcatgactggggtgaagtcgtaacaaggtagccgtatcggaaggtgcggctggatcacctcctttca

SEQ ID NO: 19 - DNA, 1059 bp, Thauera sp. strain SND5
atgtccctcctctccgcactccgtctcaccgcctggcgccaggtcgtccccaccctcgcgctcgccacgctcgccggctggctggccgagcgcctgcatctgccgctgccgtggatgatcgggccgctgtttgccgtggccggcgggcgcatggccgacatcgacctgcgtccgctccccggcgggcgccagaccgggcaatgggcgatcggcgccgcgctcggcctgtacttcagcccggaggtggtggcgcaactcgccttgcatgcaccgctggtggtcgccgccgcgctcggctcgctggtgctcggcatgggctacgccttcatcatgttgcgtctggcccgggtcgatacgccgaccgccttcttcgccgcgctgccgggcggcgcctcggagatggccaaccttgccgaccgctacggcggcgcggtggatcgcatcgccgcggctcacgccatccgggtgatgatggtggtgtcggtgctgcccttcgcgctcaccttcgccggtgtgcatggcacggacctctacgtgccgctggcaaaggcggtggatgcccggcacttcccgctgctgatcggggcgagcctgctcggggtgagcgcgttcaagctgctgcgcatcgccaacgcctgggtgctcgggccgctgctcggcgtggcggtggcaacgctggccggcgtgccgctgtcggcgctgccggcctgggtggtcaatggcggtcagttgctgatcggctgcgcccttggttgccggttttcgcgcgagttcttccgtgccgcaccgcttttcatggcggtcgcagggctgaccgcggcgatgtcgatcgtgctcgcctttgctttcgctgcgctgctcggcctggtctcggcggtccctttgcccacgctggcgctggcgaccgcgcccggtggggtgtcggagatgtgcatcaccgccaaggtgctgcagctcggcgtgccgctcgtcaccgtgtgtcacgtgttgcgcgtggtggtgctgaccgtgggcgcgcagtggagcttcggcgtcttccgccgcctggtggcggcgtga

SEQ ID NO: 20 - DNA, 2082 bp, Thauera sp. strain SND5
atgtacacgccccgctcgccgctatcctacccgcccgaacacttcctcaaccgcgaactgaccctgctgcagttccagcgccgcgtgctcgcccaggccgccgatgccaccgttccgctgctcgagcgcctgcgcttcctgtgcatcgtgtcgagcaacctcgacgagttcttcgagatccgcgtctccggcatcaaggagcagatccgcatcggcagccgcaagtcgggcgaggacggcatcgcgccggccgacctgctcgagcgcgtcagcgacgaggtgcacgagatcatttcggagcagtacgaactgctcaacgacgacatcctccccgcgctcgaaaaggaaggcgtggtgttcctgcgccgcagcctgtggacggaggcgcagcgcgcctggatccacgactacttcatgcgcgaggtgatgccggtgctgacgccgatcggtctcgaccccgcccaccccttcccgcgcgtgctcaacaagagcctgaacttcgccgtcgaactcgagggccgcgatgccttcggccgcgactcgggggccgcgatcgttcaggcgccgcgcgcgctgccgcgggtgatccggctgccgcgcgaactgtgcgcccaggaatacacctacgtgttcctgtcctcggtgctgcacgcccacgtcggcgagctgttcaccggcatgaacgtgctcggctgctatcagttccgcgtcacccgcaactccgacctgttcgtcgacgaggaagaggtcaaggacctgcgcgcctcgctcaagggcgaactgcaacagcgccacttcggcgacgccgtgcgcctggaggttgcggacaactgctcggacgagatggcgagcttcctgctgcagcactttcacctgacgcccgacgacttgtaccgcacgccgggcatcgtcaacctggtgcgcctgatgcaggtgcccgactgggtcgagcggcccgatctcaaatacacgcccttcgttcccggcctgcccaaggcgctcgacaagcgccgccagatcttcactgcgatccgcagcggcgacatcctgctccaccaccccttccagagcttcggcccggtgatcgagctgctgcgtgcggccgcggacgatcccaaggtgctggcgatcaagatgaccgtgtatcgcaccggcaccgactcggtgctgatggagcacctcgcccgcgccgcgcagaagggcaaggaggtgaccgtcgtgctggagctgatggcgcgcttcgacgaggaagccaacatcaccctcgccaaccgcctggaagaagtcggcgcgcacgtcgtgtacggcgtgttcggctacaagactcacgccaagctgctgatgatcgtgcgccgcgaggacgacggcctgcgccgctacgtgcacatgggcaccggcaactaccacccgcgcaccacccgcttctacaccgacttcggcctgctgacctgcaaccaggagatcggcgaggacgtcgccgcggtgttcaagcagctgaccgggctgggcaccgccaccgaactgcgccacctgtggcaagcaccgttcaccctgcagcccaacgtggtcgcggccatccgtcgcgaggcggaaatcgcccgcgaggggcggcgcggccgcgtgatcgcgaagatgaatgcgctgctcgaaccggaaaccatcgaggcgctatacgaggcctcgcaggccggggtcgagatcgacctgatcgtgcgcggcccctgcgcgctgcgcccgggggtaccgggtctgtccgacaacatccgcgtgcgctcggtggtcggccgcttcctcgagcaccaccgcatcttccacttccatgccgacggcgaagaccggatgtacctgtcgagcgcggactggatggaccgcaacttcttccgccgcatcgagatcgccttcccggtgctcgacccgcgcctcaagcgccgcgtcatgaaggaaggcctgcgcccctacctcggcgacaactgccaggcctgggacatgcagtcggatggcaagtaccgccgcaagcacccgcgcagcatccgccgggcggcacagctgatcctgctgaaggaactggcggcgagcagctga

SEQ ID NO: 21 - DNA, 1188 bp, Thauera sp. strain SND5
atgagcaaggccgaagaccttcaagcccccgccaccgacactgcgcagacgcctgctgccggcgcgaagaaaccaccgcgcaggaagcccgcgagccgccgtgcgcgagacggtgacaccgcgcccgagctgagccccgaaggcatcgagcacttcgaggtcgagcaggccatcgcggccgccgagagctccaagctcgtcgccgtctccgacatcctcaagaccgagaccggacgcgaccccaggcgcgcggcacgcgccctcgacgccatcctcgccggcgcctcgcccgacgacatccatgtgctgcgtcgcgcgctgatgcgcgccgacaccggcaccaccggcgccgtcgccaacccggacgacgagctcgcgcccgactggcgccagggcggctacccctatcgcaacctgatgtcgcgcaaggcctacgagaagcagaagtaccgcctgcaggtggagctgctcaagctccaggcctgggtcaaggacaccggccagcgcgtcgtgatcctgttcgaaggtcgcgacgccgccggcaagggcggcaccatcaagcgcttcatggaacacctgaacccgcgcggtgcgcgtgtggtggcgctggaaaagccctccgagatggagcgcggccagtggtacttccagcgctacatccagcacctgcccaccgccggcgaaatcgtgctcttcgaccgctcctggtacaaccgcgccggggtggagcgcgtgatgggtttctgcaccaacgacgagtacaacgagttcatgcgccaggtgcccgagttcgagcgcaacctggtgcgcagcggcatccacctgatcaagttctggttctcggtcagccgcgaggagcagcgccgccgcttcaaggagcgcgaatcgcacccgctcaagcagtggaagctgtcgccgatcgacctcgcctcgctggacaaatgggacgactacaccaaggccaaggaggcgatgttcttctacaccgacacggcggattcgccatggacggtggtgaagtccgattgcaagaagcgcgcgcgcctcaacgcactgcgctacgtgctgcacaagctgccctatgccaacaaggacatcgagcgcatcggcccgctcgacccgctgctggtcggtcgcgccaacgtggtgtatgagcgcggcgagaaggacgccgttccgctgctctga

(19) United States
(12) Patent Application Publication
(10) Pub. No.: US 2024/0351926 A1
(43) Pub. Date: Oct. 24, 2024

(54) NUTRIENTS REMOVAL VIA SIMULTANEOUS NITRIFICATION AND DENITRIFICATION COUPLING WITH PHOSPHATE ACCUMULATION

(71) Applicant: NATIONAL UNIVERSITY OF SINGAPORE, Singapore (SG)

(72) Inventors: Jianzhong HE, Singapore (SG); Qingkun WANG, Singapore (SG)

(73) Assignee: NATIONAL UNIVERSITY OF SINGAPORE, Singapore (SG)

(21) Appl. No.: 18/683,448

(22) PCT Filed: Aug. 13, 2021
(86) PCT No.: PCT/SG2021/050475

§ 371 Date: Feb. 13, 2024

(51) Int. Cl. C02F 3/30; C12N 9/02

(52) U.S. Cl. CPC C02F 3/302; C02F 3/308; C12N 9/0083; C12Y 114/99039

(57) ABSTRACT

The present invention relates to methods for water treatment. in particular using a novel Thauera species that is capable of removing nitrogen via simultaneous nitrification and denitrification. The novel Thauera species may be further capable of phosphate accumulation in the cells. The invention also relates to an isolated Thauera sp. strain SND5 (accession number: CGMCC 21549). The invention further relates to reactors for use in carrying out the methods as described herein and bioprocesses that are carried out using said reactor.

### FIELD OF THE INVENTION

The invention is in the field of water treatment, in particular water treatment using an isolated bacterial strain.

### SEQUENCE LISTING

The instant application contains a Sequence Listing which has been submitted electronically in ASCII format and is hereby incorporated by reference in its entirety. Said ASCII copy, created on Jul. 12, 2021, is named 76075PCT_Sequence Listing_ST25.txt and is 13,632 bytes in size.

### BACKGROUND OF THE INVENTION

Nitrogen and phosphorus are essential nutrients for growth and reproduction of plants and animals. Overuse of fertilizers and feed containing these elements can threaten drinking water supplies as well as negatively impact recreational and fishing activities in natural waterbodies exposed to excess nutrients. Because of the risks associated with high levels of nitrogen and phosphorus in the environment, it is essential to limit the concentration of these nutrients in water, such as wastewater treatment plant (WWTP) effluents. Biological methods using microorganisms are considered to be the most efficient way to remove nutrients from wastewater. However, current biological methods are associated with many disadvantages, such as slow rates and low efficiencies of nutrients removal, greenhouse gas emission as an end product, and strict requirement of aerobic and anoxic conditions and thereby the requirement of separate reactors.

Therefore, there is a need to provide alternative biological methods for water treatment, novel bacterial strains to be used in these methods, reactors to carry out these methods, and bioprocesses to be carried out in these reactors.

### SUMMARY

In one aspect, there is provided a method for water treatment comprising a step of contacting the water with a Thauera species that is capable of removing nitrogen via simultaneous nitrification and denitrification (SND) in the presence of a carbon source.

In another aspect, there is provided an isolated Thauera sp. strain SND5 (accession number: CGMCC 21549).

In another aspect, there is provided a reactor for use in carrying out the method as described herein.

In yet another aspect, there is provided a bioprocess that is carried out using the reactor as described herein.

### DEFINITIONS

The term “water treatment” refers to a process used to remove contaminants and undesirable components, or reduces their concentration, in the water. Such contaminants and undesirable components may be, for example, excess nutrients. Water treatment improves the quality of water to make it appropriate for a specific end-use, such as drinking, industrial water supply, irrigation, river flow maintenance, water recreation, or being safely returned to the environment.

As used herein, the term “fixed nitrogen” refers to nitrogen fixed as ammonium, nitrate, or nitrite. Ammonium may be used interchangeably with ammonia.

The term “metabolic intermediates” or “intermediates” refer to molecules that are the precursors or metabolites of biologically significant molecules. Intermediates may be produced during the nitrogen removal. For example, an intermediate may be hydroxylamine (NH₂OH) produced during the biological removal of ammonium (NH₄⁺).

As used herein, the term “simultaneous nitrification and denitrification (SND)” refers to a bioprocess where multiple fixed nitrogen species are simultaneously removed. The fixed nitrogen species may refer to ammonium, nitrate, or nitrite. For example, SND may refer to the simultaneous removal of ammonium and nitrate, the simultaneous removal of ammonium and nitrite, or the simultaneous removal of ammonium, nitrate and nitrite. SND does not refer to the resulting products or specific pathway.

As used herein, the term “heterotrophic ammonium oxidation” refers to a nitrogen removal mechanism where ammonium is converted to nitrogen gas with hydroxylamine produced as an intermediate. Heterotrophic ammonium oxidation proceeds as NH₄⁺→NH₂OH→N₂. Heterotrophic ammonium oxidation is different from conventional ammonium oxidation which proceeds as NH₄⁺→NH₂OH→NO→NO₂⁻.

As used herein, the term “denitrifying” or “denitrification” refers to a process where nitrate (NO₃⁻) and/or nitrite (NO₂⁻) are reduced to nitrogenous gas, such as nitrogen gas (N₂), and nitrous oxide (N₂O).

“Phosphate-accumulating organisms (PAOs)” are a group of bacteria that, under certain conditions, facilitate the removal of phosphorus from water by accumulating the phosphorus within the cell as polyphosphate. PAOs that are capable of removing nitrogen from the water are known as denitrifying phosphate-accumulating organisms (DPAOs).

“Polyhydroxyalkanoates (PHAs)” are a family of polyhydroxyesters of 3-, 4-, 5- and 6-hydroxyalkanoic acids, which are produced by a variety of bacterial species under nutrient-limiting conditions with excess carbon. They may be accumulated intracellularly and serve as a carbon and energy storage compound.

The “removal efficiency” of a nutrient is defined as the ratio of the amount of a nutrient being removed over the total amount of a nutrient present in the water. The removal efficiency of ammonium is calculated as (C_{NH4+(0)}−C_{NH4+(t)})/C_{NH4+(0)}, wherein C_{NH4+(0)} is defined as the concentration of ammonium in the influent (before treatment), and C_{NH4+(t)} is defined as the concentration of ammonium in the effluent (after treatment). The removal efficiency of nitrite is calculated as (C_{NO2-(0)}−C_{NO2-(t)})/C_{NO2-(0)}, wherein C_{NO2-(0)} is defined as the concentration of nitrite in the influent (before treatment), and C_{NO2-(t)} is defined as the concentration of nitrite in the effluent (after treatment). The removal efficiency of nitrate is calculated as (C_{NO3-(0)}−C_{NO3-(t)})/C_{NO3-(0)}, wherein C_{NO3-(0)} is defined as the concentration of nitrate in the influent (before treatment), and C_{NO3-(t)} is defined as the concentration of nitrate in the effluent (after treatment). The removal efficiency of total nitrogen is calculated as (C_{TN(0)}−C_{TN(t)})/C_{TN(0),} wherein C_{TN(0)} is defined as the concentration of total nitrogen in the influent (before treatment), C_{TN(t)} is defined as the concentration of total nitrogen in the effluent (after treatment), and total nitrogen includes ammonium, nitrite, nitrate, and organic nitrogen. The removal efficiency of total phosphate is calculated as (C_{Total Phosphate(0)}−C_{Total Phosphate(t)})/C_{Total Phosphate(0)}, wherein C_{Total Phosphate(0)} is defined as the concentration of total phosphate in the influent (before treatment), C_{Total Phosphate(t)} is defined as the concentration of total phosphate in the effluent (after treatment), and total phosphate includes phosphate (PO₄³⁻), hydrogen phosphate (HPO₄²⁻), and dihydrogen phosphate (H₂PO₄⁻). The removal efficiency of total phosphorus is calculated as (C_{TP(0)}−C_{TP(t)})/C_{TP(0)}, wherein C_{TP(0)} is defined as the concentration of total phosphorus in the influent (before treatment), C_{TP(t)} is defined as the concentration of total phosphorus in the effluent (after treatment), and total phosphorus includes phosphate, hydrogen phosphate, dihydrogen phosphate, and organic phosphorus.

The term “substantially”, when used with reference to the nutrient removal efficiency, refers an efficiency that is at least about 65%. For example, a nutrient is “substantially” removed if its removal efficiency is at least about 65%, at least about 70%, at least about 80%, at least about 90%, or about 100%.

The “removal rate” of a nutrient is defined as the speed of removing a nutrient from the water. The removal rate of ammonium is calculated as (C_{NH4+(0)}−C_{NH4+(t)})/t, wherein C_{NH4+(0)} is defined as the concentration of ammonium in the influent (before treatment), C_{NH4+(t)} is defined as the concentration of ammonium in the effluent (after treatment), and t is defined as the treatment duration. The removal rate of nitrite is calculated as (C_{NO2-(0)}−C_{NO2-(t)})/t, wherein C_{NO2-(0)} is defined as the concentration of nitrite in the influent (before treatment), C_{NO2-(t)} is defined as the concentration of nitrite in the effluent (after treatment), and t is defined as the treatment duration. The removal rate of nitrate is calculated as (C_{NO3-(0)}−C_{NO3-(t)})/t, wherein C_{NO3-(0)} is defined as the concentration of nitrate in the influent (before treatment), C_{NO3-(t)} is defined as the concentration of nitrate in the effluent (after treatment), and t is defined as the treatment duration. The removal rate of total nitrogen is calculated as (C_{TN(0)}−C_{TN(t)})/t, wherein C_{TN(0)} is defined as the concentration of total nitrogen in the influent (before treatment), C_{TN(t)} is defined as the concentration of total nitrogen in the effluent (after treatment), t is defined as the treatment duration, and total nitrogen includes ammonium, nitrite, nitrate, and organic nitrogen. The removal rate of total phosphate is calculated as (C_{Total Phosphate(0)}−C_{Total Phosphate(t)})/t, wherein C_{Total Phosphate(0)} is defined as the concentration of total phosphate in the influent (before treatment), C_{Total Phosphate(t)} is defined as the concentration of total phosphate in the effluent (after treatment), t is defined as the treatment duration, and total phosphate includes phosphate, hydrogen phosphate, and dihydrogen phosphate. The removal rate of total phosphorus is calculated as (C_{TP(0)}−C_{TP(t)}/t, wherein C_{TP(0)} is defined as the concentration of total phosphorus in the influent (before treatment), C_{TP(t)} is defined as the concentration of total phosphorus in the effluent (after treatment), t is defined as the treatment duration, and total phosphorus includes phosphate, hydrogen phosphate, dihydrogen phosphate, and organic phosphorus.

The term “dissolved oxygen (DO) level” is defined as the amount of oxygen that is present in the water.

The term “microoxic condition” refers to a condition where the dissolved oxygen (DO) level is between about 0.5 ppm and about 1.0 ppm inclusive. For example, under the microoxic condition, the DO level may be about 0.5 ppm, about 0.6 ppm, about 0.7 ppm, about 0.8 ppm, about 0.9 ppm, or 1.0 ppm.

The term “sequencing batch reactor (SBR)” is a reactor to carry out an activated sludge type wastewater treatment process involving an acration step. Sequencing batch reactor process may include multiple phases, such as filling, reaction, settling, idle, and decanting phases.

A reactor may involve multiple phases, such as filling, reaction, settling, idle, and decanting phases. During a “filling phase”, the reactor is to be filled with water to be treated through an open inlet valve. The filling phase may also be known as the medium introduction phase. A “reaction phase” is the phase where a microorganism reacts to remove the nutrients from the water. The reaction phase may comprise an aerobic stage, an anoxic stage, or a combination thereof. At the aerobic stage, aeration with stirring is applied to create aerobic condition in the reactor. At the anoxic stage, no acration is applied and hence the reactor is under anoxic condition. Stirring may be applied for homogeneity. During a “settling phase”. no aeration or stirring is applied and sedimentation takes place. During a “decanting phase”, the treated water is to be removed from the reactor through an open outlet valve. The decanting phase may also be known as the medium withdrawal phase. During an “idle phase”, no action is applied. The idle phase takes place between the filling and decanting phases and acts as a buffer in time. The idle phase may be omitted when the multiple phases occur in a single reactor.

The term “solids retention time (SRT)” refers to the time that the solid fraction of the wastewater spends in the treatment system. SRT is an important parameter for wastewater treatment reactors, relating to growth rate of the microorganism and to effluent concentrations. SRT may be calculated as the effective volume of the reactor divided by the volume of solids removed from the reactor per day. The solids refer to the biomass. The solids removed from the reactor may be used for sampling or discarded.

As used herein, the term “about”, in the context of, but not limited to, nutrient removal efficiency or rate, DO level, or operation parameters of a reactor, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### BRIEF DESCRIPTION OF THE DRAWINGS

The invention will be better understood with reference to the detailed description when considered in conjunction with the non-limiting examples and the accompanying drawings, in which:

FIG. 1 Phylogeny of Thauera sp. strain SND5. A maximum-likelihood tree of the 16S rRNA gene from representative members of the Thauera genus was inferred using MEGA7. Accession numbers of representative strains are indicated before the taxonomic designation. The final tree is supported by 1000 bootstraps; values for nodes present in >50% of the trees are indicated.

FIG. 2 Effects of (A) carbon source and (B) C/N ratio on nitrate removal efficiency and cell growth of Thaeura sp. strain SND5. Data shown are mean of triplicate experiments; error bars indicate S.D. and in some cases may be smaller than symbols.

FIG. 3 Batch tests of conventional denitrification by Thauera sp. strain SND5. Denitrification of (A) nitrate, (B) nitrite, and (C) a nitrate and nitrite mixture by strain SND5 generates nitrous oxide without accumulation of nitrite as an intermediate. Data shown are mean of triplicate experiments; error bars indicate S.D. and in some cases may be smaller than symbols.

FIG. 4 PCR amplification of functional denitrification and nitrification genes using genomic DNA from Thacura sp. strain SND5 as template. Lane M, DNA marker in 100 bp. (A) Lane 1, narG gene PCR product; Lane 2, nirS gene PCR product; Lane 3, norB gene PCR product; Lane 4, nosZ gene PCR product; Lane 5, ppk1 gene PCR product in positive control; Lane 6, ppk1 gene PCR product in SND5; (B) Lane 7, Lane 8 and Lane 9, no bands can be found for amoA/B and HAO genes in SND5; Lane 10-12, amoA gene PCR product in positive controls; Lane 13-14, amoB gene PCR product in positive controls; Lane 16-17, HAO gene PCR product in positive controls.

FIG. 5 Batch tests of heterotrophic nitrification by strain SND5 in closed systems with the detection of nitrogenous gases. Changes in concentrations of heterotrophic oxidation of (A) ammonium and (B) hydroxylamine. Data shown are mean of triplicate experiments; error bars indicate S.D. and in some cases may be smaller than symbols.

FIG. 6 Aerobic denitrification and ammonium oxidation by Thauera sp. strain SND5 in open systems. Cultures were amended with (A) nitrate; (B) nitrite; (C) ammonium and (D) nitrate and nitrite. Data shown are mean of triplicate experiments; error bars indicate S.D. and in some cases may be smaller than symbols.

FIG. 7 Culture SND5 can perform simultaneous nitrification and denitrification (SND) in open systems. Cultures were amended with (A) nitrate and ammonium; (B) nitrate, nitrite and ammonium and (C) nitrite and ammonium. Data shown are mean of triplicate experiments; error bars indicate S.D. and in some cases may be smaller than symbols.

FIG. 8 Nitrate denitrification by culture SND5 with intracellular polyhydroxybutyrate (PHB) as a carbon source. Data shown are mean of triplicate experiments; error bars indicate S.D. and in some cases may be smaller than symbols.

FIG. 9 Phosphate accumulation by culture SND5 during polyhydroxybuterate (PHB) catabolismorresponding decreases in concentrations of PHB and (A) NO₃⁻, (B) NO₂⁻, and (C) O₂ amended as an electron acceptor following complete depletion of lactate. Control cultures not amended with electron acceptor (D) continued to accumulate PHB. Data shown are mean of triplicate experiments; error bars indicate S.D. and in some cases may be smaller than symbols.

### DETAILED DESCRIPTION OF THE PRESENT INVENTION

In one aspect, the present invention refers to a method for water treatment comprising a step of contacting the water with a Thauera species that is capable of removing nitrogen via simultaneous nitrification and denitrification (SND) in the presence of a carbon source.

The water to be treated using the method as disclosed herein may be wastewater or non-wastewater. Suitable types of wastewater may include, but are not limited to, household wastewater, domestic wastewater, municipal wastewater, industrial wastewater, and agricultural wastewater. The industrial wastewater may be of different origins. In one example, the industrial wastewater may be of food-processing origins, such as wastewater from the cereal starch production industry, dairy products production industry, meat centers, sugar production industry, livestock food products production industry, livestock farming, meat products production industry, meat ham and sausage production industry, fishery paste product production industry, or fishery food products production industry. In another example, the industrial wastewater may be from organic or inorganic chemical production industry. In yet another example, the industrial wastewater may be from refineries or petrochemical industry. The agricultural wastewater may include, but is not limited to, wastewater from piggery, dairy or fish farms. The municipal or domestic wastewater may include, but is not limited to, blackwater or greywater. Black water may be from, for example, toilets and kitchen sinks. Greywater may be from, for example, showers, baths, whirlpool tubs, washing machines, dishwashers, and sinks other than the kitchen sink. The water to be treated may contain diverse volatile fatty acids, such as formic acid, acetic acid, and lactic acid. In one example, the water to be treated is municipal landfill leachates. In another example, the water to be treated is piggery wastewater.

The water to be treated typically contains one or more nutrients that need to be removed. In some examples, the nutrients may be present in excess.

One example of the nutrients to be removed is nitrogen. The source of nitrogen in the water may be fixed nitrogen. Examples of suitable nitrogen sources include, but are not limited to, ammonium (NH₄⁺), nitrate (NO₃⁻), and nitrite (NO₂⁻). In one example, the nitrogen source comprises ammonium (NH₄⁺). In another example, the nitrogen source comprises nitrate (NO₃⁻). In another example, the nitrogen source comprises nitrite (NO₂⁻). In another example, the nitrogen source comprises ammonium (NH₄⁺) and nitrate (NO₃⁻). In another example, the nitrogen source comprises ammonium (NH₄⁺) and nitrite (NO₂⁻). In another example, the nitrogen source comprises nitrate (NO₃⁻) and nitrite (NO₂⁻). In yet another example, the nitrogen source comprises ammonium (NH₄⁺), nitrate (NO₃⁻) and nitrite (NO₂⁻).

Using the method as disclosed herein, the nitrogen is removed by contacting the water with a Thauera species that is capable of removing nitrogen via SND.

With a Thauera species that is capable of removing nitrogen via SND, ammonium (NH₄⁺) may be removed alone or simultaneously with nitrate (NO₃⁻) and/or nitrite (NO₂⁻) depending on the availability of nitrogen sources in the water.

During the process of ammonium (NH₄⁺) removal, metabolic intermediates may be produced. In one example, hydroxylamine (NH₂OH) may be produced as an intermediate. The hydroxylamine produced may by removed immediately without any accumulation. In another example, nitrite (NO₂⁻) may not be produced as an intermediate during the removal of ammonium (NH₄⁺). It is known that accumulation of nitrite (NO₂⁻) inhibits the process of nitrogen removal and compromises the removal efficiency. Therefore, compared to conventional methods for ammonium (NH₄⁺) removal where nitrite (NO₂⁻) is produced and accumulated, the method as disclosed herein may improve the nitrogen removal efficiency.

Ammonium (NH₄⁺) may be converted to nitrogenous gas as an end product and released into the atmosphere. The nitrogenous gas may be nitrogen gas or nitrogen oxides. Suitable examples of nitrogen oxides may include, but are not limited to, nitrous oxide (N₂O), and nitric oxide (NO). The ammonium nitrogen may be removed by being converted to an end product selected from the group consisting of nitrogen (N₂), nitrous oxide (N₂O), nitric oxide (NO), and combinations thereof. In a preferred example, ammonium (NH₄⁺) is removed without producing nitrous oxide (N₂O) as an end product. In a more preferred example, ammonium (NH₄⁺) is removed by producing nitrogen gas (N₂) as the only end product. It is known that nitrogen oxides are air polluting chemical compounds. In particular, nitrous oxide (N₂O) is a greenhouse gas which has far-ranging environmental and health effects. Therefore, it would be appreciated that reducing or eliminating the production of nitrogen oxides, such as nitrous oxide (N₂O), is advantageous in water treatment.

In a preferred example, ammonium (NH₄⁺) is removed via heterotrophic ammonium oxidation proceeding as NH₄⁺→NH₂OH→N₂. Ammonium (NH₄⁺) is converted to hydroxylamine (NH₂OH) followed by nitrogen gas (N₂) without producing other intermediates.

Similar to ammonium (NH₄⁺), nitrate (NO₃⁻) and/or nitrite (NO₂⁻) may be removed alone or simultaneously with ammonium (NH₄⁺) depending on the availability of nitrogen sources in the water.

Nitrate (NO₃⁻) and/or nitrite (NO₂⁻) in the water may be removed via denitrification. In one example, the nitrogen source in the water comprises nitrate (NO₃⁻). In another example, the nitrogen source in the water comprises nitrite (NO₂⁻). In yet another example, the nitrogen source in the water comprises nitrate (NO₃⁻) and nitrite (NO₂⁻).

During the process of denitrification, nitrite (NO₂⁻) may be produced as intermediate. Using the method as disclosed herein, nitrite (NO₂⁻) produced may be immediately removed, resulting no accumulation of nitrite (NO₂⁻), and thus improving the nitrogen removal rate. In a preferred example, nitrite (NO₂⁻) and/or nitrate (NO₃⁻) are removed without accumulation of nitrite (NO₂⁻). Nitrate (NO₃⁻) and/or nitrite (NO₂⁻) may be converted to an end product selected from nitrogen gas (N₂), nitrous oxide (N₂O), nitric oxide (NO), or combinations thereof. In one example, the end product comprises nitrous oxide (N₂O).

In one example, nitrate (NO₃⁻) and/or nitrite (NO₂⁻) are removed by conversion to nitric oxide (NO) followed by nitrous oxide (N₂O). Other intermediates and end products may be produced during the removal process. In a preferred example, nitrate (NO₃⁻) and/or nitrite (NO₂⁻) can be removed via denitrification proceeding as NO₃⁻→NO₂⁻→NO→N₂O→N₂ without other intermediates produced.

To enable the nitrogen removal, the presence of a carbon source in the water to be treated is required. The carbon source in the water may be organic or inorganic carbon. In a preferred example, the carbon source comprises organic carbon. Examples of organic carbon may include, but are not limited to, lactate, acetate, succinate, formate, and glucose. In a preferred example, the carbon source comprises one or more selected from lactate, acetate, and succinate. In one example, the carbon source comprises acetate. In another example, the carbon source comprises succinate. In another example, the carbon source comprises acetate and succinate. In a more preferred example, the carbon source comprises lactate. In another example, the carbon source comprises lactate and acetate. In another example, the carbon source comprises lactate and succinate. In yet another example, the carbon source comprises lactate, acetate, and succinate.

Conventionally, the carbon content relative to nitrogen content, i.e., C/N ratio, is an important determinant of nitrogen removal efficiency in a biological wastewater treatment system due to the competition between heterotrophs for carbon sources. It is particularly important for the denitrification process. When the carbon supply is limited, the nitrogen removal process is compromised. When the carbon is in excess, the nitrogen removal process is also compromised and the environment in the water favors the development of filamentous organisms which ultimately destroy the nutrient removal system.

The method as disclosed herein may not be limited by the C/N ratio. The Thauera species as used in the method may produce and store intracellular carbon during the catabolismof an initial carbon source. Such Thauera species may be a denitrifying phosphate-accumulating organism (DPAO). The carbon may be stored intracellularly as polyhydroxyalkanoates (PHAs). Examples of PHAs may include, but are not limited to, poly-3-hydroxybutyrate (PHB), poly-3-hydroxybutyrate-co-4-hydroxybutyrate (P(3-HB-co-4-HB)), poly-3-hydroxybutyrate-co-valerate (PHBV), or polyhydroxybutyrate-co-hexanoate (PHBH). In a preferred example, the carbon is stored intracellularly as PHB.

Such intracellularly stored carbon can be used to sustain the cell activities, such as denitrification process, when external carbon sources are depleted. As such, the cell activities, such as denitrification process, may continue without the requirement of additional carbon sources. For example, the Thauera species produces and stores PHB during catabolism of lactate catabolismand subsequently utilizes the intracellular PHB as a carbon source for nitrate denitrification when lactate is depleted. In one example, nitrogen is removed without the requirement of additional carbon sources after depletion of the carbon source.

It would be understood that the method as disclosed herein is suitable to treat water with a wide range of C/N ratios. In one example, the water has a low C/N ratio that is about 5 or lower than about 5. In another example, the water has a high C/N ratio that is about 10 or higher than about 10. In yet another example, the water has a C/N ratio between about 5 and about 10. The water may have a C/N ratio between about 1 and about 40 inclusive, between about 2 and about 39 inclusive, between about 3 and about 38 inclusive, between about 4 and about 37 inclusive, between about 5 and about 36 inclusive, between about 6 and about 35 inclusive, between about 7 and about 34 inclusive, between about 8 and about 33 inclusive, between about 9 and about 32 inclusive, between about 10 and about 31 inclusive, between about 11 and about 30 inclusive, between about 12 and about 29 inclusive, between about 13 and about 28 inclusive, between about 14 and about 27 inclusive, between about 15 and about 26 inclusive, between about 16 and about 25 inclusive, between about 17 and about 24 inclusive, between about 18 and about 25 inclusive, between about 19 and about 24 inclusive, between about 20 and about 23 inclusive, or between about 21 and about 22 inclusive.

A low C/N ratio may be, for example, about 5, about 4.5, about 4, about 3.5, about 3, about 2.5, about 2, about 1.5, about 1, or about 0.5. In one example, the C/N ratio is between about 1 and about 5 inclusive. In another example, the C/N ratio is about 5. Examples of water with a low C/N ratio may include, but are not limited to, industrial wastewater that is rich in inorganic nitrogen, normal municipal wastewater.

A high C/N ratio may be, for example, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, about 30, about 30.5, about 31, about 31.5, about 32, about 32.5, about 33, about 33.5, about 34, about 34.5, about 35, about 35.5, about 36, about 36.5, about 37, about 37.5, about 38, about 38.5, about 39, about 39.5, about 40. In one example, the C/N ratio is between about 12 and about 32 inclusive. In another example, the C/N ratio is about 10. In another example, the C/N ratio is about 15. Examples of water with a high C/N ratio may include, but are not limited to, piggery wastewater, municipal landfill leachates, wastewater from fermentation, wastewater from digestion, and wastewater containing livestock manures.

A C/N ratio between about 5 and about 10 may be, for example, about 5.5, about 6, about 6.5, about 7, about 7.5, about 8, about 8.5, about 9, about 9.5. In one example, the C/N ratio is about 7.5. In yet another example, the C/N ratio is between about 8 and about 10.

In a preferred example, the C/N ratio is between about 1 and about 5, about 5, about 7.5, about 10, about 15, between about 8 and about 10, or between about 12 and about 32.

According to the method as disclosed herein, total nitrogen may be substantially removed with an efficiency of at least about 65%. The total nitrogen removal efficiency may be, for example, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%. In a preferred example, the total nitrogen is substantially removed with an efficiency of at least about 90%. In one example, the total nitrogen includes ammonium, nitrite, nitrate, and organic nitrogen.

Ammonium (NH₄⁺) may be substantially removed by the method of the invention with an efficiency of at least about 65%. The ammonium removal efficiency may be, for example, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%. In a preferred example, ammonium (NH₄⁺) is substantially removed with an efficiency of at least 90%.

Nitrate (NO₃⁻) may be substantially removed by the method of the present invention with an efficiency of at least about 65%. The nitrate removal efficiency may be, for example, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%. In a preferred example, nitrate (NO₃⁻) is substantially removed with an efficiency of at least 95%.

Nitrite (NO₂⁻) may be substantially removed with an efficiency of at least about 65%. The nitrite removal efficiency may be, for example, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%. In a preferred example, nitrite (NO₂⁻) is substantially removed with an efficiency of at least about 95%.

According to the method as disclosed herein, total nitrogen may be removed at a rate between about 3.00 mg-N/L/h and about 10.00 mg-N/L/h inclusive. For example, the total ammonium nitrogen may be removed at a rate of at least about 3.10 mg-N/L/h, at least about 3.20 mg-N/L/h, at least about 3.30 mg-N/L/h, at least about 3.40 mg-N/L/h, at least about 3.50 mg-N/L/h, at least about 3.60 mg-N/L/h, at least about 3.70 mg-N/L/h, at least about 3.80 mg-N/L/h, at least about 3.90 mg-N/L/h, at least about 4.00 mg-N/L/h, at least about 4.10 mg-N/L/h, at least about 4.20 mg-N/L/h, at least about 4.30 mg-N/L/h, at least about 4.40 mg-N/L/h, at least about 4.50 mg-N/L/h, at least about 4.60 mg-N/L/h, at least about 4.70 mg-N/L/h, at least about 4.80 mg-N/L/h, at least about 4.90 mg-N/L/h, at least about 5.00 mg-N/L/h, at least about 5.10 mg-N/L/h, at least about 5.20 mg-N/L/h, at least about 5.30 mg-N/L/h, at least about 5.40 mg-N/L/h, at least about 5.50 mg-N/L/h, at least about 5.60 mg-N/L/h, at least about 5.70 mg-N/L/h, at least about 5.80 mg-N/L/h, at least about 5.90 mg-N/L/h, at least about 6.00 mg-N/L/h, at least about 6.10 mg-N/L/h, at least about 6.20 mg-N/L/h, at least about 6.30 mg-N/L/h, at least about 6.40 mg-N/L/h, at least about 6.50 mg-N/L/h, at least about 6.60 mg-N/L/h, at least about 6.70 mg-N/L/h, at least about 6.80 mg-N/L/h, at least about 6.90 mg-N/L/h, at least about 7.00 mg-N/L/h, at least about 7.10 mg-N/L/h, at least about 7.20 mg-N/L/h, at least about 7.30 mg-N/L/h, at least about 7.40 mg-N/L/h, at least about 7.50 mg-N/L/h, at least about 7.60 mg-N/L/h, at least about 7.70 mg-N/L/h, at least about 7.80 mg-N/L/h, at least about 7.90 mg-N/L/h, at least about 8.00 mg-N/L/h, at least about 8.10 mg-N/L/h, at least about 8.20 mg-N/L/h, at least about 8.30 mg-N/L/h, at least about 8.40 mg-N/L/h, at least about 8.50 mg-N/L/h, at least about 8.60 mg-N/L/h, at least about 8.70 mg-N/L/h, at least about 8.80 mg-N/L/h, at least about 8.90 mg-N/L/h, at least about 9.00 mg-N/L/h, at least about 9.10 mg-N/L/h, at least about 9.20 mg-N/L/h, at least about 9.30 mg-N/L/h, at least about 9.40 mg-N/L/h, at least about 9.50 mg-N/L/h, at least about 9.60 mg-N/L/h, at least about 9.70 mg-N/L/h, at least about 9.80 mg-N/L/h, at least about 9.90 mg-N/L/h, or at least about 10.00 mg-N/L/h. In a preferred example, total nitrogen may be removed at a rate of at least about 8.00 mg-N/L/h. In one example, the total nitrogen includes ammonium, nitrite, nitrate, and organic nitrogen.

Ammonium (NH₄⁺) may be removed at a rate between about 1.00 mg-N/L/h and about 4.00 mg-N/L/h inclusive. For example, the ammonium nitrogen may be removed at a rate of at least about 1.00 mg-N/L/h, at least about 1.10 mg-N/L/h, at least about 1.20 mg-N/L/h, at least about 1.30 mg-N/L/h, at least about 1.40 mg-N/L/h, at least about 1.50 mg-N/L/h, at least about 1.60 mg-N/L/h, at least about 1.70 mg-N/L/h, at least about 1.80 mg-N/L/h, at least about 1.90 mg-N/L/h, at least about 2.00 mg-N/L/h, at least about 2.10 mg-N/L/h, at least about 2.20 mg-N/L/h, at least about 2.30 mg-N/L/h, at least about 2.40 mg-N/L/h, at least about 2.50 mg-N/L/h, at least about 2.60 mg-N/L/h, at least about 2.70 mg-N/L/h, at least about 2.80 mg-N/L/h, at least about 2.85 mg-N/L/h, at least about 2.90 mg-N/L/h, at least about 3.00 mg-N/L/h, at least about 3.10 mg-N/L/h, at least about 3.20 mg-N/L/h, at least about 3.30 mg-N/L/h, at least about 3.40 mg-N/L/h, at least about 3.50 mg-N/L/h, at least about 3.60 mg-N/L/h, at least about 3.70 mg-N/L/h, at least about 3.80 mg-N/L/h, at least about 3.90 mg-N/L/h, or at least about 4.00 mg-N/L/h. In a preferred example, ammonium (NH₄⁺) may be removed at a rate of at least about 2.30 mg-N/L/h.

Nitrate (NO₃⁻) may be removed at a rate between about 1.00 mg-N/L/h and about 4.00 mg-N/L/h. For example, the ammonium nitrogen may be removed at a rate of at least about 1.00 mg-N/L/h, at least about 1.10 mg-N/L/h, at least about 1.20 mg-N/L/h, at least about 1.30 mg-N/L/h, at least about 1.40 mg-N/L/h, at least about 1.50 mg-N/L/h, at least about 1.60 mg-N/L/h, at least about 1.70 mg-N/L/h, at least about 1.80 mg-N/L/h, at least about 1.90 mg-N/L/h, at least about 1.98 mg-N/L/h, at least about 2.00 mg-N/L/h, at least about 2.10 mg-N/L/h, at least about 2.20 mg-N/L/h, at least about 2.30 mg-N/L/h, at least about 2.40 mg-N/L/h, at least about 2.50 mg-N/L/h, at least about 2.60 mg-N/L/h, at least about 2.70 mg-N/L/h, at least about 2.80 mg-N/L/h, at least about 2.90 mg-N/L/h, at least about 3.00 mg-N/L/h, at least about 3.10 mg-N/L/h, at least about 3.20 mg-N/L/h, at least about 3.30 mg-N/L/h, at least about 3.40 mg-N/L/h, at least about 3.50 mg-N/L/h, at least about 3.60 mg-N/L/h, at least about 3.70 mg-N/L/h, at least about 3.80 mg-N/L/h, at least about 3.90 mg-N/L/h, or at least about 4.00 mg-N/L/h. In a preferred example, nitrate (NO₃⁻) may be removed at a rate of at least about 1.80 mg-N/L/h nitrate.

Nitrite (NO₂⁻) may be removed at a rate between about 1.00 mg-N/L/h and about 4.00 mg-N/L/h inclusive. For example, the ammonium nitrogen may be removed at a rate of at least about 1.00 mg-N/L/h, at least about 1.10 mg-N/L/h, at least about 1.20 mg-N/L/h, at least about 1.30 mg-N/L/h, at least about 1.40 mg-N/L/h, at least about 1.50 mg-N/L/h, at least about 1.60 mg-N/L/h, at least about 1.70 mg-N/L/h, at least about 1.80 mg-N/L/h, at least about 1.90 mg-N/L/h, at least about 2.00 mg-N/L/h, at least about 2.10 mg-N/L/h, at least about 2.20 mg-N/L/h, at least about 2.30 mg-N/L/h, at least about 2.40 mg-N/L/h, at least about 2.42 mg-N/L/h, at least about 2.50 mg-N/L/h, at least about 2.60 mg-N/L/h, at least about 2.70 mg-N/L/h, at least about 2.80 mg-N/L/h, at least about 2.90 mg-N/L/h, at least about 3.00 mg-N/L/h, at least about 3.10 mg-N/L/h, at least about 3.20 mg-N/L/h, at least about 3.30 mg-N/L/h, at least about 3.40 mg-N/L/h, at least about 3.50 mg-N/L/h, at least about 3.60 mg-N/L/h, at least about 3.70 mg-N/L/h, at least about 3.80 mg-N/L/h, at least about 3.90 mg-N/L/h, or at least about 4.00 mg-N/L/h. In a preferred example, nitrite (NO₂⁻) may be removed at a rate of at least about 2.00 mg-N/L/h.

The Thauera species can simultaneously remove multiple fixed nitrogen selected from ammonium (NH₄⁺), nitrate (NO₃⁻), and nitrite (NO₂⁻).

In one example, ammonium (NH₄⁺) and nitrate (NO₃⁻) are simultaneously removed. In another example, ammonium (NH₄⁺) and nitrite (NO₂⁻) are simultaneously removed. In another example, nitrate (NO₃⁻), and nitrite (NO₂⁻) are simultaneously removed. In a preferred example, ammonium (NH₄⁺), nitrate (NO₃⁻), and nitrite (NO₂⁻) are simultaneously removed. In a more preferred example, ammonium (NH₄⁺), nitrate (NO₃⁻), and nitrite (NO₂⁻) are simultaneously removed without accumulation of nitrite (NO₂⁻). In another preferred example, ammonium (NH₄⁺), nitrate (NO₃⁻), and nitrite (NO₂⁻) are simultaneously removed at a rate of at least about 2.30, about 1.80, about 2.00 mg-N/L/h. respectively.

Another example of nutrients to be removed is phosphorus. The source of phosphorus in the water may be, but is not limited to, phosphate (PO₄³⁻), hydrogen phosphate (HPO₄²⁻), and dihydrogen phosphate (H₂PO₄⁻). In one example, the phosphorous source in the water comprises phosphate (PO₄³⁻). In another example, the phosphorus source in the water comprises hydrogen phosphate (HPO₄²⁻). In another example, the phosphorus source in the water comprises dihydrogen phosphate (H₂PO₄⁻). In another example, the phosphorus source in the water comprises phosphate (PO₄³⁻) and hydrogen phosphate (HPO₄²⁻). In another example, the phosphorus source in the water comprises phosphate (PO₄³⁻) and dihydrogen phosphate (H₂PO₄⁻). In another example, the phosphorus source in the water comprises hydrogen phosphate (HPO₄²⁻) and dihydrogen phosphate (H₂PO₄⁻). In yet another example, the phosphorus source in the water comprises phosphate (PO₄³⁻), hydrogen phosphate (HPO₄²⁻) and dihydrogen phosphate (H₂PO₄⁻).

In the method as disclosed herein, the phosphorus in the water is removed by contacting the water with a Thauera species that is further capable of removing phosphorous.

In one example, phosphorus is removed via phosphate accumulating where the Thauera species absorbs the phosphate from the water and stores it intracellularly. It is understood that an electron acceptor is required during phosphate accumulating. Suitable examples of electron acceptors may include, but are not limited to, nitrate (NO₃⁻), nitrite (NO₂⁻), and oxygen. The electron acceptor may be from the nitrogen source. In one example, the electron acceptor comprises nitrate (NO₃⁻). In another example, the electron acceptor comprises nitrite (NO₂⁻). In yet another example, the electron acceptor comprises both nitrate (NO₃⁻) and nitrite (NO₂⁻). The electron acceptor may be from sources other than the nitrogen source. For example, the electron acceptor comprises oxygen.

In one example, the electron acceptor comprises nitrate (NO₃⁻) and/or nitrite (NO₂⁻). It would be understood phosphorus is removed via denitrifying phosphate accumulating when the electron acceptor comprises nitrate (NO₃⁻) and nitrite (NO₂⁻). In one example, phosphorus is removed via denitrifying phosphate accumulating.

As described in the foregoing, the Thauera species used in the method as disclosed herein may be capable of storing intracellular carbon and subsequent utilization under nutrient-limiting condition. As such, phosphorous removal via phosphate accumulation may continue when the initial carbon source is depleted and no additional carbon sources are required. It would be appreciated that it is particularly advantageous in treating water that has low carbon content. In one example, phosphorus is removed without the requirement of additional carbon source after depletion of the initial carbon source.

The carbon source in the water may be organic or inorganic carbon. In a preferred example, the carbon source comprises organic carbon. Examples of organic carbon may include, but are not limited to, lactate, acetate, succinate, formate, and glucose. In a preferred example, the carbon source comprises one or more selected from lactate, acetate, and succinate. In one example, the carbon source comprises acetate. In another example, the carbon source comprises succinate. In another example, the carbon source comprises acetate and succinate. In a more preferred example, the carbon source comprises lactate. In another example, the carbon source comprises lactate and acetate. In another example, the carbon source comprises lactate and succinate. In yet another example, the carbon source comprises lactate, acetate, and succinate.

According to the method as disclosed herein, total phosphorus may be removed with an efficiency between about 15% and about 70% inclusive. For example, the total phosphorus removal efficiency may be at least about 15%, at least about 20%, at least about 25%, at least about 30%, at least about 35%, at least about 40%, at least about 45%, at least about 50%, at least about 55%, at least about 60%, at least about 61%, at least about 62%, at least about 63%, at least about 64%, at least about 65%, at least about 66%, at least about 67%, at least about 68%, at least about 69%, or at least about 70%. In one example, total phosphorus may be removed with an efficiency of at least 60%. In another example, total phosphorus is substantially removed with an efficiency of at least 65%. In yet another example, total phosphorus is substantially removed with an efficiency of at least 70%. In one example, the total phosphorus includes phosphate, hydrogen phosphate, dihydrogen phosphate, and organic phosphorus. The concentration of total phosphorus may be measured by a total phosphorus measurement kit.

Total phosphate may be removed with an efficiency between about 20% and 70% inclusive. For example, the total phosphate removal efficiency may be at least about 20%, at least about 25%, at least about 30%, at least about 35%, at least about 40%, at least about 45%, at least about 50%, at least about 55%, at least about 60%, at least about 65%, or at least about 70%. In a preferred example, the total phosphate is substantially removed with an efficiency of at least about 65%. In one example, the total phosphate includes phosphate, hydrogen phosphate, and dihydrogen phosphate.

According to the method as disclosed herein, total phosphorus may be removed at a rate between about 5.00 mg-P/L/h and about 15.00 mg-P/L/h inclusive. For example, the total phosphorus may be removed at a rate of at least about 5.00 mg-P/L/h, at least about 5.50 mg-P/L/h, at least about 6.00 mg-P/L/h, at least about 6.50 mg-P/L/h, at least about 7.00 mg-P/L/h, at least about 7.50 mg-P/L/h, at least about 8.00 mg-P/L/h, at least about 8.50 mg-P/L/h, at least about 9.00 mg-P/L/h, at least about 9.50 mg-P/L/h, at least about 10.00 mg-P/L/h, at least about 10.50 mg-P/L/h, at least about 11.00 mg-P/L/h, at least about 11.50 mg-P/L/h, at least about 12.00 mg-P/L/h, at least about 12.50 mg-P/L/h, at least about 13.00 mg-P/L/h, at least about 13.30 mg-P/L/h, at least about 13.50 mg-P/L/h, at least about 14.00 mg-P/L/h, at least about 14.50 mg-P/L/h, or at least about 15.00 mg-P/L/h. In a preferred example, total phosphorus may be removed at a rate of at least about 10 mg-P/L/h. In a more preferred example, total phosphorus is removed at a rate of at least about 13.3 mg-P/L/h. In one example, the total phosphorus includes phosphate, hydrogen phosphate, dihydrogen phosphate, and organic phosphorus. The concentration of total phosphorus may be measured by a total phosphorus measurement kit.

Total phosphate may be removed with a rate between about 4.00 mg-P/L/h and about 13.00 mg-P/L/h inclusive. For example, the total phosphate may be removed at a rate of at least about 4.00 mg-P/L/h, at least about 4.50 mg-P/L/h, at least about 5.00 mg-P/L/h, at least about 5.50 mg-P/L/h, at least about 6.00 mg-P/L/h, at least about 6.50 mg-P/L/h, at least about 7.00 mg-P/L/h, at least about 7.50 mg-P/L/h, at least about 8.00 mg-P/L/h, at least about 8.50 mg-P/L/h, at least about 9.00 mg-P/L/h, at least about 9.50 mg-P/L/h, at least about 10.00 mg-P/L/h, at least about 10.50 mg-P/L/h, at least about 11.00 mg-P/L/h, at least about 11.50 mg-P/L/h, at least about 12.00 mg-P/L/h, at least about 12.50 mg-P/L/h, or at least about 13.00 mg-P/L/h. In a preferred example, the total phosphate is removed at a rate of at least about 12.5 mg-P/L/h. In one example, the total phosphate includes phosphate, hydrogen phosphate, and dihydrogen phosphate.

As disclosed herein, the method may be carried out at a wide range of dissolved oxygen (DO) level. Suitable DO level may range from about 0.1 ppm to about 2.0 ppm. For example, the DO level may be about 0.1 ppm, about 0.2 ppm, about 0.3 ppm, about 0.4 ppm. about 0.5 ppm, about 0.6 ppm, about 0.7 ppm, about 0.8 ppm, about 0.9 ppm, about 1.0 ppm, about 1.1 ppm, about 1.2 ppm, about 1.3 ppm, about 1.4 ppm, about 1.5 ppm, about 1.6 ppm, about 1.7 ppm, about 1.8 ppm, about 1.9 ppm, or about 2.0 ppm.

In one example, nitrogen from different sources are simultaneously removed at a

DO level of about 1.0 ppm or between about 1.0 ppm and about 2.0 ppm. For example, the DO level may be about 1.0 ppm, about 1.1 ppm, about 1.2 ppm, about 1.3 ppm, about 1.4 ppm, about 1.5 ppm, about 1.6 ppm, about 1.7 ppm, about 1.8 ppm, about 1.9 ppm, or about 2.0 ppm. In one example, the nitrogen source comprises ammonium (NH₄⁺) and nitrate (NO₃⁻). In another example, the nitrogen source comprises ammonium (NH₄⁺) and nitrite (NO₂⁻). In yet another example, the nitrogen source comprises ammonium (NH₄⁺), nitrate (NO₃⁻) and nitrite (NO₂⁻).

In another example, ammonium is removed at a DO level between about 1.0 ppm and about 2.0 ppm inclusive. For example, the DO level may be about 1.0 ppm, about 1.1 ppm, about 1.2 ppm, about 1.3 ppm, about 1.4 ppm, about 1.5 ppm, about 1.6 ppm, about 1.7 ppm, about 1.8 ppm, about 1.9 ppm, or about 2.0 ppm.

In another example, nitrate and/or nitrite are removed at a DO level below about 0.2 ppm, or below about 0.1 ppm.

In yet another example, phosphate is removed at a DO level between about 0.5 ppm and 2.0 ppm inclusive. For example, the DO level may be about 0.5 ppm, about 0.6 ppm, about 0.7 ppm, about 0.8 ppm, about 0.9 ppm, about 1.0 ppm, about 1.1 ppm, about 1.2 ppm, about 1.3 ppm, about 1.4 ppm, about 1.5 ppm, about 1.6 ppm, about 1.7 ppm, about 1.8 ppm, about 1.9 ppm, or about 2.0 ppm. In one example, the DO level is about 2.0 ppm.

The method as disclosed herein may also be carried out under microoxic condition at a DO level of between about 0.5 ppm and 1.0 ppm inclusive. For example, the DO level may be about 0.5 ppm, about 0.6 ppm, about 0.7 ppm, about 0.8 ppm, about 0.9 ppm, or about 1.0 ppm. In a preferred example, the DO level may be about 0.8 ppm, between about 0.8 ppm and about 1.0 ppm, or about 1.0 ppm. The nutrients to be removed may be selected from ammonium, nitrate, nitrite, phosphate, hydrogen phosphate, dihydrogen phosphate, and combinations thereof.

With the method disclosed herein, nitrogen and phosphorous may be removed via a single bioprocess. The removal of nitrogen and phosphorous as a single bioprocess alleviates the competition for carbon sources between different heterotrophs in the water and thus improves nutrient removal efficiency.

As disclosed herein, the Thauera species may express various genes that enable the bioprocesses involved in water treatment.

In one example, the Thauera species expresses one or more ppk genes, which encode polyphosphate kinase. Polyphosphate kinase is known to control phosphate uptake and accumulation. In one example, the ppk genes encode the same protein. The ppk genes may be selected from ppk1, ppk2, or a combination thereof. In one example, the Thauera species expresses ppk1 gene. In another example, the Thauera species expresses ppk2 gene. In a preferred example, the Thauera species expresses both ppk1 and ppk2 genes. In one example, the ppk1 gene has a DNA sequence as set forth in SEQ ID NO: 20. In another example, the ppk2 gene has a DNA sequence as set forth in SEQ ID NO: 21.

In another example, the Thauera species may express an amo gene, which encodes an ammonia monooxygenase that catalyzes a non-conventional ammonium oxidation pathway. A person skilled in the art would understand that the conventional ammonium oxidation pathway proceeds as NH₄⁺→NH₂OH→NO→NO₂⁻. A non-conventional ammonium oxidation pathway may be any ammonium oxidation pathway that proceeds differently from the conventional pathway proceeding as NH₄⁺→NH₂OH→NO→NO₂⁻. In a preferred example, the amo gene encodes an ammonia monooxygenase that catalyzes the heterotrophic ammonium oxidation proceeding as NH₄⁺→NH₂OH→N₂. In a more preferred example, the amo gene has a DNA sequence as set forth in SEQ ID NO: 19.

In yet another example, the Thauera species may express both ppk and amo genes.

In a preferred example, the Thauera species comprises a partial DNA sequence of 16S rRNA gene as set forth in SEQ ID NO: 17. In a more preferred example, the Thauera species comprises a full DNA sequence of 16S rRNA gene as set forth in SEQ ID NO: 18.

In a more preferred example, the Thauera species is Thauera sp. strain SND5 with accession number CGMCC 21549. The Thauera sp. strain was isolated and named as SND5, and deposited at the China General Microbiological Culture Collection Centre (CGMCC) (Institute of Microbiology Chinese Academy of Sciences, No. 1 West Beichen Road, Chaoyang District, Beijing 100101, China), an international depository authority, on 28 Dec. 2020 under the accession number CGMCC 21549.

As disclosed herein, the Thauera species may be used with other bacterial species. For example, the Thauera species may be present in a culture containing other bacteria.

In another aspect, there is provided an isolated Thauera sp. strain SND5 with accession number CGMCC 21549.

There is also provided an isolated Thauera sp. strain SND5 with accession number CGMCC 21549 for use in carrying out the methods as described herein. There is further provided use of an isolated Thauera sp. strain SND5 with accession number CGMCC 21549 in carrying out the methods as described herein.

In another aspect, there is provided a reactor for use in carrying out the methods as described herein. There is also provided use of a reactor in carrying out the methods as described herein.

Examples of suitable reactors may include, but are not limited to, an activated sludge reactor, a biofilm reactor, a sequencing batch reactor, a membrane bioreactor, and upflow anaerobic sludge bed (UASB). In a preferred example, the reactor comprises a sequencing batch reactor. In one example, the sequencing batch reactor is a lab-scale sequencing batch reactor.

The reactor may be first inoculated with a culture comprising the Thauera sp. strain as disclosed herein.

Following the inoculation, the reactor may be operated with cycles comprising multiple phases. The multiple phases may comprise one or more phases selected from filling. reaction, settling, idle, and decanting phases. Filling and decanting phases may also be known as medium introduction and withdrawal phases, respectively. In one example, the multiple phases comprise filling, reaction, settling, idle, and decanting phases. In a preferred example, each cycle comprising the multiple phases proceeds as filling phase→reaction phase→settling phase→decanting phase→idle phase.

During the filling phase, the reactor is filled with the water to be treated. The reactor may be filled through an open inlet valve. The filling phase may last for a period of time, for example, about 10 minutes, about 15 minutes, about 20 minutes, about 25 minutes, or about 30 minutes. In a preferred example, the filling phase lasts for 15 minutes.

The reaction phase may take place under aerobic condition, anoxic condition, or a combination thereof. The aerobic condition may be created by applying aeration with stirring. The anoxic condition may be created by terminating the acration. Stirring may be applied to create homogeneity in the reactor. In one example, the reaction phase takes place under acrobic and anoxic conditions. In a preferred example, the reaction phase takes place under sequential aerobic and anoxic conditions. In another example, the reaction phase takes place by alternating acrobic and anoxic conditions in cycles.

In one example, both aerobic and anoxic conditions are to favor the biological reactions in the reactor. It would be understood that it is advantageous that bacterial growth and metabolism are encouraged under both aerobic and anoxic conditions.

The reaction phase may last for a period of time, for example, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours. In one example, the reaction phase lasts for 22 hours. The anoxic condition may also last for a period of time, for example, about 15 hours, about 16 hours, about 17 hours, about 18 hours, about 19 hours, about 20 hours, about 21 hours, or about 22 hours. In one example, the anoxic condition lasts for about 22 hours, about 19 hours, about 18 hours, or about 16 hours. In a preferred example, the anoxic condition lasts for about 16 hours. The acrobic condition may also last for a period of time, for example, about 0 hour, about 1 hour, about 2 hours, about 3 hours, about 4 hours, about 5 hours, about 6 hours, or about 7 hours. In one example, the aerobic condition lasts for about 0 hour, about 3 hours, about 4 hours, or about 6 hours. In a preferred example, the acrobic condition lasts for about 6 hours. In a more preferred example, the reaction phase lasts for about 22 hours with about 16 hours of anoxic condition and about 6 hours of aerobic condition.

Under the anoxic condition, the DO level may be lower than about 0.20 ppm. For example, the DO level may be about 0.20 ppm, about 0.15 ppm, about 0.10 ppm, or 0.05 ppm. Under the aerobic condition, the DO level may be between about 0.80 ppm and about 1.00 ppm inclusive. For example, the DO level may be about 0.80 ppm, 0.85 ppm, 0.90 ppm, 0.95 ppm, or 1.00 ppm.

During the settling phase, acration and stirring are terminated and biomass is allowed to settle for a period of time. Sedimentation is allowed to take place at this phase. The settling time may be, for example, about 1 hour, about 2 hours, about 3 hours, about 4 hours, or about 5 hours. In a preferred example, the settling time is about 1 hour.

During decanting phase, the treated water is to be removed from the reactor. The treated water may be removed through an open outlet valve. The decanting phase may last for a period of time, for example, about 10 minutes, about 15 minutes, about 20 minutes, about 25 minutes, or about 30 minutes. In a preferred example, the decanting phase lasts for about 15 minutes.

During the idle phase, no action is applied. The idle phase may take place between the filling and decanting phases and act as a buffer in time. The idle phase may last for, for example, about 0.5 hour, about 1 hour, about 1.5 hour, about 2 hours, or about 3 hours. In a preferred example, the idle phase lasts for about 0.5 hour. In another example, the idle phase is omitted.

It is understood that the quantity of water to be introduced or removed depends on the effective volume of the reactor. For example, the quantity of water to be introduced or removed is a maximum of 3 L if the effective volume of the reactor is 3 L.

According to the reactor as disclosed herein, the multiple phases may be run in cycle. For example, the cycle may be an about 12 hour, about 13 hour, about 14 hour, about 15 hour, about 16 hour, about 17 hour, about 18 hour, about 19 hour, about 20 hour, about 21 hour, about 22 hour, about 23 hour, about 24 hour, about 25 hour, about 26 hour, about 27 hour, about 28 hour, about 29 hour, or about 30 hour cycle. In a preferred example, the cycle is an about 24 hour cycle.

During the cycles, the volume of water to be introduced at the filling phase may be balanced with the volume of water to be removed at the decanting phase. The SRT may be kept at, for example, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days. In one example, the SRT is kept at about 14 days, or about 28 days. In a preferred example, the SRT is kept at about 28 days. In some examples, filling phase takes place immediately after decanting phase.

The reactor may be operated at a C/N ratio between about 1 and about 20 inclusive. For example, the C/N ratio may be about 1, about 2, about 3, about 4, about 5, about 6, about 7, about 8, about 9, about 10, about 11, about 12, about 13, about 14, about 15, about 16, about 17, about 18, about 19, or about 20. In one example, the C/N ratio is about 1, or about 2. In a preferred example, the C/N ratio is about 2.

In a preferred example, the reactor is operated in 24 hour cycle, wherein the anoxic time is about 16 hours, the aerobic time is about 6 hours, the settling time is about 1 hour, the idle phase lasts for about 0.5 hours, the SRT is maintained at about 28 days

In yet another aspect, there is provided a bioprocess that is carried out using the reactor as described herein.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

### Materials and Methods

### Medium and Culture Conditions

A mineral salts medium with a pH value of 7.2˜7.4 amended with different supplements was used for enrichment and isolation of denitrifying bacteria, as well as for simultancous nitrification and denitrification (SND), denitrifying phosphate accumulation and hydroxylamine oxidation experiments. The mineral salts medium contained (per liter): 1.0 g NaCl, 0.5 g MgCl₂, 0.2 g KH₂PO₄, 0.3 g KCl, 0.015 g CaCl₂ and 0.5% of trace element solution that contained (per liter): 1.5 mg FeCl₂, 0.19 mg CoCl₂, 0.1 mg MnCl₂, 0.07 mg ZnCl₂, 0.006 mg H₃BO₃, 0.036 mg Na₂MoO₄, 0.024 mg NiCl₂, 0.002 mg CuCl₂. For enrichment and isolation of denitrifying bacteria (denitrifying medium), the mineral salts medium was amended with a carbon source-lactate (C/N ratio of ˜7.5), and approximately 80 mg-N/L of either nitrate or nitrite. For SND experiments (SND medium), the mineral salts medium was amended with lactate (C/N of ˜7.5), and ˜80 mg-N/L of nitrate, nitrite and ammonium each with a dissolved oxygen (DO) concentration of ˜1.0 mg/L throughout the tests. For denitrifying phosphate-accumulation experiment (DPAO medium), the mineral salts medium was amended with lactate (˜90 mg/L) and ˜15 mg-N/L of either nitrate or nitrite. For ammonium oxidation experiments (ammonium oxidation medium), the mineral salts medium was amended with ˜80 mg-N/L of NH₄⁺. For hydroxylamine oxidation experiments (hydroxylamine oxidation medium), the mineral salts medium was amended with ˜7 mg-N/L of NH₂OH. Cultures and experiments were agitated at 120 r/min unless stated otherwise. All media were autoclaved prior to use.

### Enrichment and Isolation

Strain SND5 was isolated from the anoxic tank of the Integrated Validation Plant (IVP), at the Ulu Pandan Water Reclamation Plant, Singapore. Activated sludge (5 mL) was transferred to a 160 mL serum bottle sealed with a butyl rubber stopper containing 45 mL sterile denitrifying medium and agitated to obtain a homogeneous suspension. After 1-2 days' incubation, 5 mL of the suspension was transferred into 45 mL of fresh medium (2^{nd} culture). Transfers (10% V/V) were repeated every 2 days until cultures were homogeneous and sediment-free. To isolate denitrifying bacteria, serial dilutions (10⁻¹-10⁻⁸) were conducted and then inoculated into agar shakes containing denitrifying medium. After 7 days' incubation at 30° C., single colonies were picked, cultivated in denitrifying medium and screened for denitrification activity.

### DNA Extraction and Molecular Analysis

Genomic DNA was extracted using the DNeasy Powersoil Kit according to the manufacturer's instructions. DNA extracts were quantified on a NanoDrop 1000 Spectrophotometer, diluted with sterile water to 5-10 ng/μl and stored at −20° C. for further analysis. The 16S rRNA genes of isolates picked from serial dilutions of the sediment-free enrichment were amplified using the universal primers 27F/1492R. The thermal cycling profile of PCR was: initial denaturation for 5 min at 95° C., followed by 30 cycles of denaturation (45 s at 95° C.), primer annealing (45 s at 55° C.), extension (60 s at 72° C.), and polyadenylation (6 min at 72° C.). PCR products were verified by electrophoresis on 1% agarose gels and purified using the QIAquick PCR Purification Kit. Purified PCR products were sequenced by Sanger sequencing using the universal primers 27F/1492R. Resulting sequences were identified using the BLAST tool from NCBI. Representative denitrifying bacterial sequences for phylogenetic analysis were retrieved from GenBank and the phylogeny of these representative denitrifying bacterial 16S rRNA genes was inferred using MEGA 7.0. Sequences were aligned using ClustalW and a maximum likelihood phylogenetic tree supported by 1000 bootstraps was constructed. The presence of nitrification (amo and hao), denitrification (nar, nir, nor and nos) and phosphate accumulating (ppk1) functional genes in genomic DNA extracted from the isolate was evaluated using gene-specific primers (Table 1 and Table 2). The thermal profile used for amplification: initial denaturation for 5 min at 96° C., followed by 30 cycles of denaturation (30 s at 96° C.), primer annealing (45 s, Table 1), and extension (90 s at 72° C.), and finally a last extension step of 6 min at 72° C. The amplified products were analyzed by electrophoresis on 1% agarose gels.

TABLE 1
Primers

| | Annealing | | SEQ |
|---|---|---|---|
| | temperature | | ID |
| Primer | (° C.) | Sequence | NO: |
| amoA-1F | 55 | GGGGTTTCTACTGGTGGT | 1 |
| amoA-2R | | CCCCTCKGSAAAGCCTTCTTC | 2 |
| amoBMf | 53 | TGGTAYGACATKAWATGG | 3 |
| amoBMr | | RCGSGGCARGAACATSGG | 4 |
| haoF1 | 54 | TGCGTGGARTGYCAC | 5 |
| haoR3 | | AGRTARGAKYSGGCAAA | 6 |
| narG1960m2F | 58 | TAYGTSGGGCAGGARAAACTG | 7 |
| narG2050m2R | | CGTAGAAGAAGCTGGTGCTGTT | 8 |
| NirS2f | 55 | TACCACCCSGARCCGCGCGT | 9 |
| NirS3r | | GCCGCCGTCRTGVAGGAA | 10 |
| cnorB2Fb | 54 | GACAARHWVTAYTGGTGGT | 11 |
| cnorB6R | | GAANCCCCANACNCCNGC | 12 |
| nosZ1527Fb | 60 | CGYTGTTCMTCGACAGCCAG | 13 |
| nosZ1622R | | CGSACCTTSTTGCCSTYGCG | 14 |
| Acc-ppk1- | 60 | TCACCACCGACGGCAAGAC | 15 |
| 254f | | | |
| Acc-ppk1- | | ACGATCATCAGCATCTTGGC | 16 |
| 1376r | | | |

TABLE 2
Degenerate bases used in the primers

| Degenerate base designation | Actual bases coded |
|---|---|
| K | G or T |
| S | C or G |
| Y | C or T |
| W | A or T |
| R | A or G |
| V | A or C or G |
| H | A or C or T |
| N | A or C or G or T |
| M | A or C |

### Nitrogen and Phosphate Removal

A single colony of strain SND5 or culture SND5 was inoculated into 80 mL denitrifying medium and cultured at 30° C. with agitation for growth. Cultures were centrifuged at 8000 rpm and washed thrice with sterilized phosphate buffered saline (PBS) and resuspended in the mineral salts medium; 5 mL of the resuspension was inoculated into either 45 mL denitrifying medium in serum bottles sealed with butyl rubber stoppers (closed systems) or SND medium in serum bottles topped with cotton-wool air filters to allow gas exchange (open systems) and incubated at 30° C. with agitation. Samples were collected at 20 min intervals for optical determination of cell density and nutrient composition (ammonium, nitrate, nitrite, and carbon). All experiments were performed in triplicate and results are presented as mean±standard deviation. Removal rates were computed during logarithmic growth phase according to the OD600 values. Optical measurements were made on a Tecan infinite M200PRO.

For phosphate accumulation experiments, SND5 cultures (5 mL) were incubated in DPAO medium in closed systems and placed at 30° C. with agitation. After 100 min, nitrate, nitrite or oxygen was amended to serve as possible electron acceptors. A negative control without amendment of an electron acceptor at 100 min was established concurrently. Samples were collected at 20 min intervals for measurement of nutrients (nitrate/nitrite/carbon), PHB and glycogen. All experiments were performed in triplicate and results are presented as mean±standard deviation.

### Nitrogen Metabolism

Strain SND5 or culture SND5 was incubated in 45 mL ammonium or hydroxylamine oxidation medium in closed systems flushed with oxygen and incubated at 30° C. with agitation. An abiotic control without bacteria was established concurrently. Headspace samples (100 μL) were collected using a gastight syringe and manually injected into a GC-TCD equipped with molecular sieve 5A column (30 m×0.53 mm×40 μm, Agilent) for detection of O₂ and N₂ with helium as a carrier gas at a flow rate of 20 mL/min.

The column and detector temperatures were 70 and 180° C., respectively. N₂O was analyzed by GC-ECD as previously described. The amount of O₂, N₂ and N₂O were calculated by applying the ideal gas law. Samples were collected at defined intervals of 2 or 3 h from SND5 cultures grown at 30° C. for optical determination of cell density. All experiments were performed in triplicate and results are presented as mean±standard deviation.

### Analytical Methods

All batch-test samples were centrifuged at 13,000 rpm to remove biomass prior to analysis. NH₄⁺, NO₂, NO₃, PO₄³⁻ and lactate ions were measured using a Metrohm IC System. Anions were measured on an Eco IC equipped with a Metrosep A Supp5 analytical column (4×250 mm) while cations were measured on a Metrohm 930 Compact IC Flex equipped with a Metrosep C6 analytical column (4×250 mm). Eluent flow rates for anion detection (3.2 mM carbonate/1.0 mM bicarbonate) and cation detection (4.8 mM nitrite acid/0.78 mM dipicolinic acid) were 0.7 and 0.9 mL/min, respectively.

The concentration of hydroxylamine was determined spectrophotometrically at 705 nm using matched 1 cm Corex cuvettes. Other reagents and procedures were as previously described. PHB and glycogen were analyzed.

### Example 1

### Identification of Strain SND5

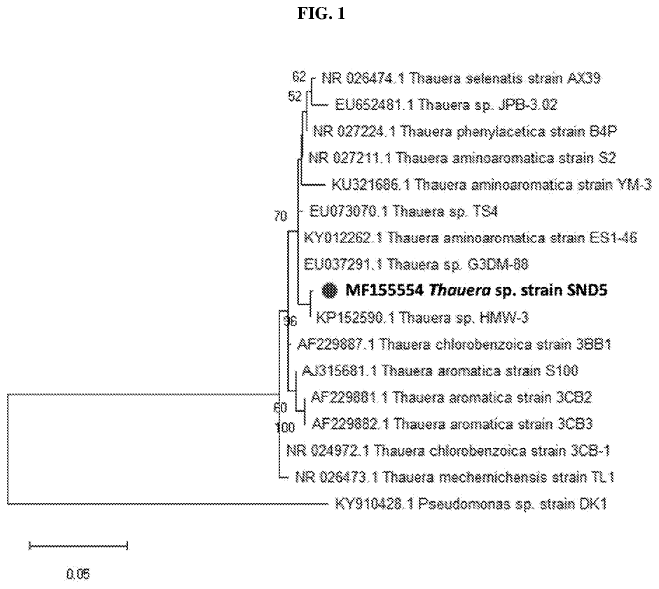

Activated sludge from an anoxic tank in a wastewater treatment plant was cultivated and transferred sequentially in a denitrifying medium containing nitrate and a carbon source—lactate. Then the serial diluted culture was inoculated to agar shakes filled with the same medium. After 48 h incubation at 30° C., creamy, convex, light-yellow, opaque colonies of approximately 2-5 mm in diameter with regular edges grew on agar shakes. The colonies were sticky and not conducive to picking. The partial 16S rRNA gene (1389 nt) of the colonies was amplified, sequenced and de-posited in GenBank under accession number MF155554. The 16S rRNA gene was most similar to members of the genus Thauera, with the highest sequence similarity (99%) to Thauera sp. HMW-3; these two forms a distinct cluster within a dendrogram (FIG. 1). Based on these results, the new isolate was determined to be a novel member of the genus Thauera and was designated SND5.

### Example 2

### Characterization of Strain SND5—Carbon Source Utilization and Optimum C/N Ratio

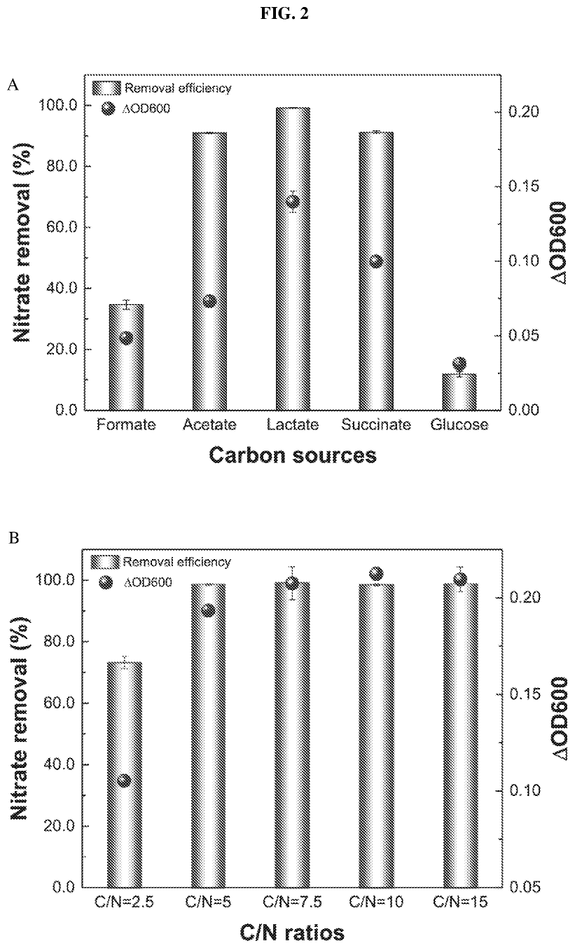

To study nitrogen removal efficiency by strain SND5, different carbon source, formate, acetate, lactate, succinate or glucose was provided at a C/N ratio of 10:1 (FIG. 2a). Among these, lactate yielded the highest nitrate removal efficiency (99.2%) and supported the greatest cell growth (ΔOD600: 0.14). Though the highest nitrate removal efficiency was supported by lactate, acetate and succinate supported comparable removal efficiencies (above 90%), indicating that strain SND5 could effectively use a wide range of carbon sources. Being able to utilize various carbon sources advantages strain SND5 to grow and function in the wastewater treatment stream where diverse VFAs, such as formic acid, acetic acid and lactic acid, are available.

Different C/N ratios with lactate as the carbon source were pro-vided to investigate the optimal ratio for strain SND5 to remove nitrate (FIG. 2b). Comparable nitrogen removal efficiencies were observed at C/N ratios of 5, 7.5, 10 and 15 (98.7%. 99.2%, 98.6% and 98.9%, respectively) within 8 hours, illustrating that increasing C/N ratios exhibited negligible impact on nitrogen removal efficiency. The wide range of C/N ratios over which strain SND5 maintained high nitrogen removal suggests that this strain might have practical applications for biological treatment of wastewater, such as piggery wastewater and municipal landfill leachates that have a C/N ratio of 8˜10 and 12˜32. respectively. Results in this study suggest that the C/N ratio is not a determining factor in denitrification mediated by strain SND5, which could reduce the amount of COD augmentation to achieve effective nitrogen removal. Thus, a C/N ratio of 7.5 was selected in the following batch tests.

### Example 3

### Characterization of Strain SND5—Assay on Nitrogen Removal Via Conventional Denitrification

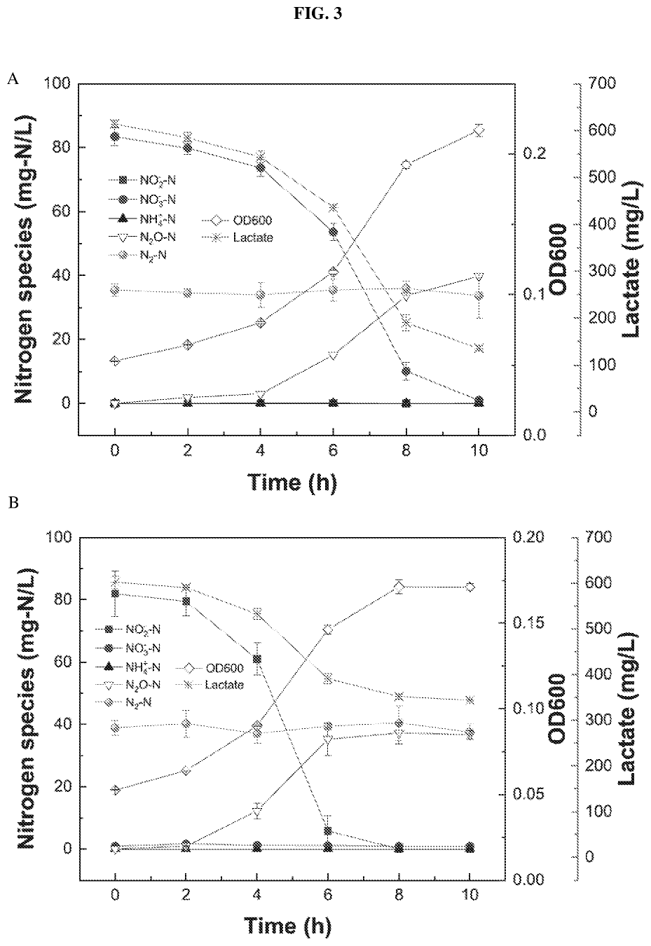

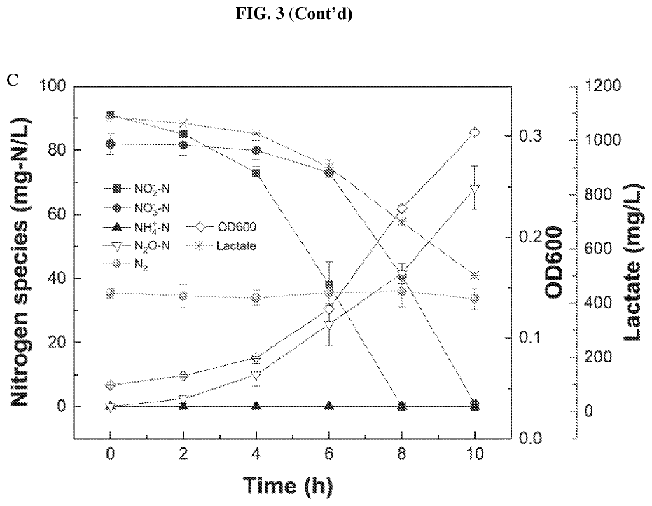

Using lactate as a carbon source and nitrate as a nitrogen source, strain SND5 removed all of the nitrate in the system (83.44 mg-N/L) after 10 h, at an average rate of 8.34 mg-N/L/h, via conventional denitrification (FIG. 3). Nitrate removal was accompanied by a rapid increase in cell number (ΔOD600: 0.16) within 8 hours, after an initial lag of 2 hours. When added as the sole nitrogen source, nitrite was removed completely within 8 hours at a higher rate (10.24 mg-N/L/h) than nitrate. Notably, nitrite removal yielded lower cell density (ΔOD600: 0.12) than did nitrate, consistent with organic carbon consumption of 478 and 258 mg/L lactate when using nitrate or nitrite, respectively (FIGS. 3a and b). Both nitrite and nitrate were denitrified simultaneously when present together, although nitrite was reduced faster than nitrate (11.36 and 8.10 mg-N/L/h, respectively; FIG. 3c); consistent with the results using either nitrite or nitrate as the sole nitrogen source.

The rate of denitrification by strain SND5 was higher than that of many previously identified denitrifying isolates, such as 2.2 mg-N/(L·h) for Klebsiella pneumonia CF-S9 1.99 mg-N/(L·h) for Pseudomonas tolaasii Y-11, and 0.93 mg-N/(L·h) for Rhodococcus sp. CPZ24, suggesting it could have practical applications for nitrate denitrification in wastewater. Notably, the difference in the rates of nitrite and nitrate denitrification resulted in no accumulation of nitrite in cultures of strain SND5, a unique property which is different from previously described denitrifying bacteria. It has previously been reported that nitrite accumulation is an inevitable consequence of nitrate denitrification and acts as an inhibitor of biological nutrients removal. Nitrite accumulation in WWTPs leads to reductions in overall nitrogen removal efficiency, as it is produced rather than nitrogenous gases as an end-product. The lack of nitrite accumulation exhibited by strain SND5 during nitrate denitrification, therefore, might provide an avenue by which nitrite inhibition in biological wastewater treatment processes could be circumvented.

### Example 4

### Nitrogen Metabolic Pathway

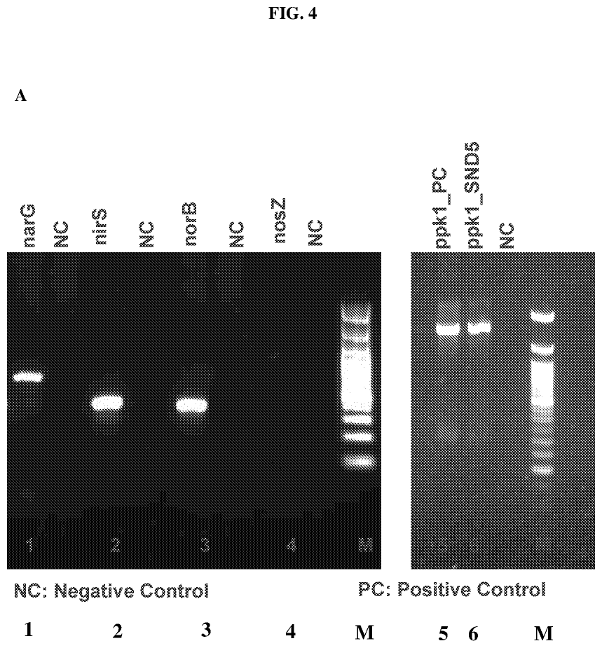

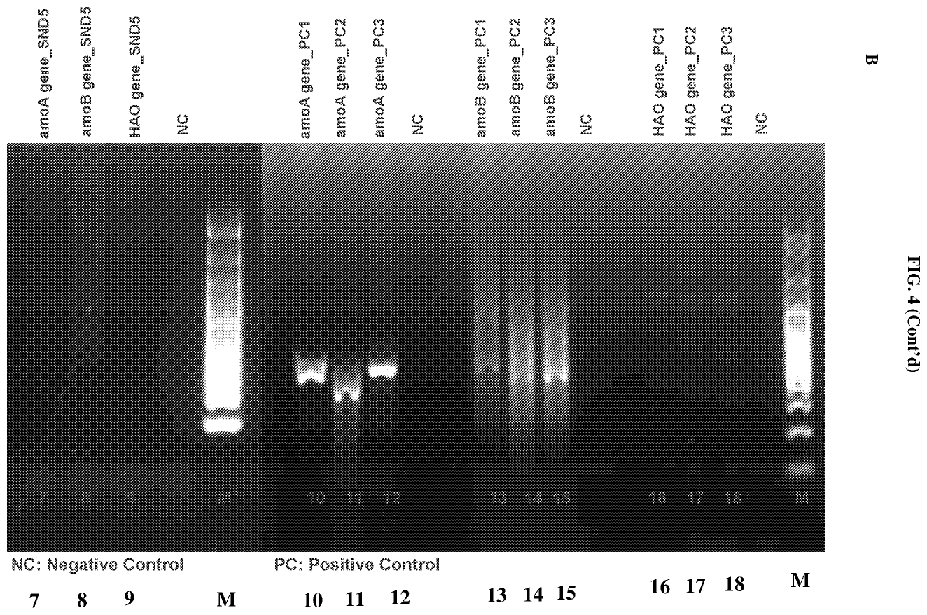

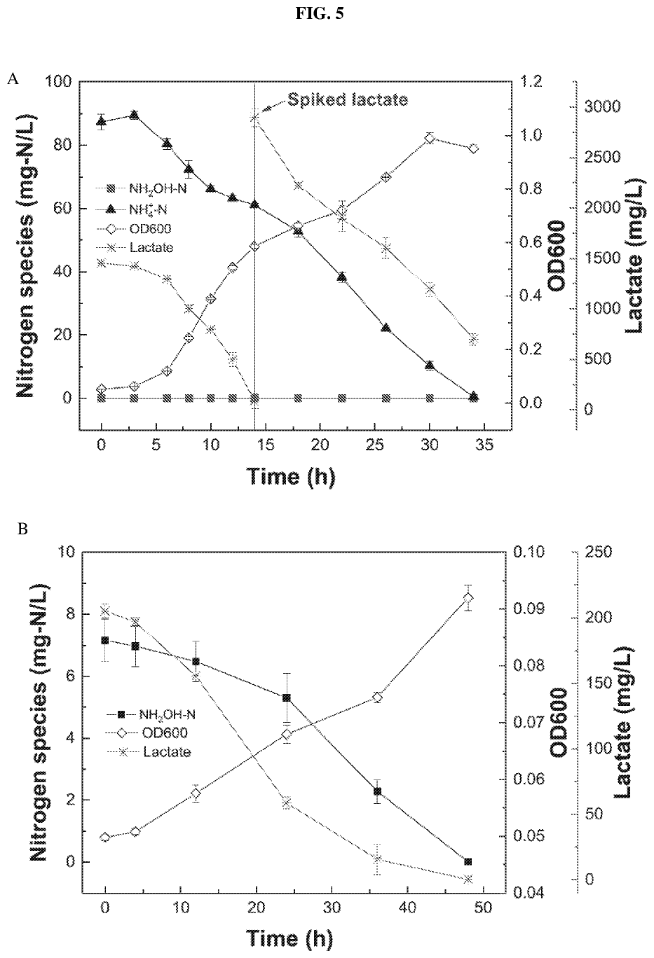

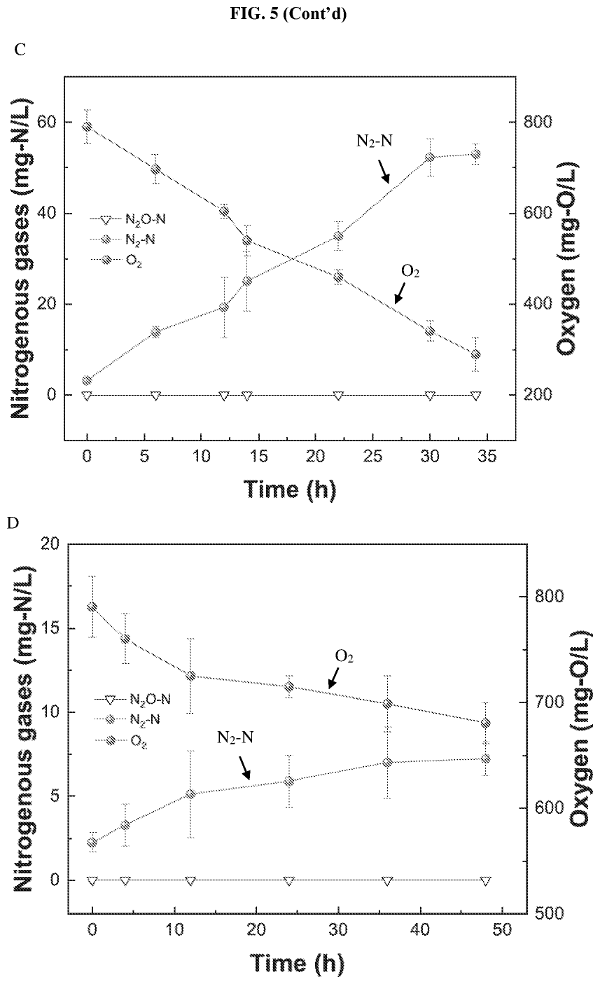

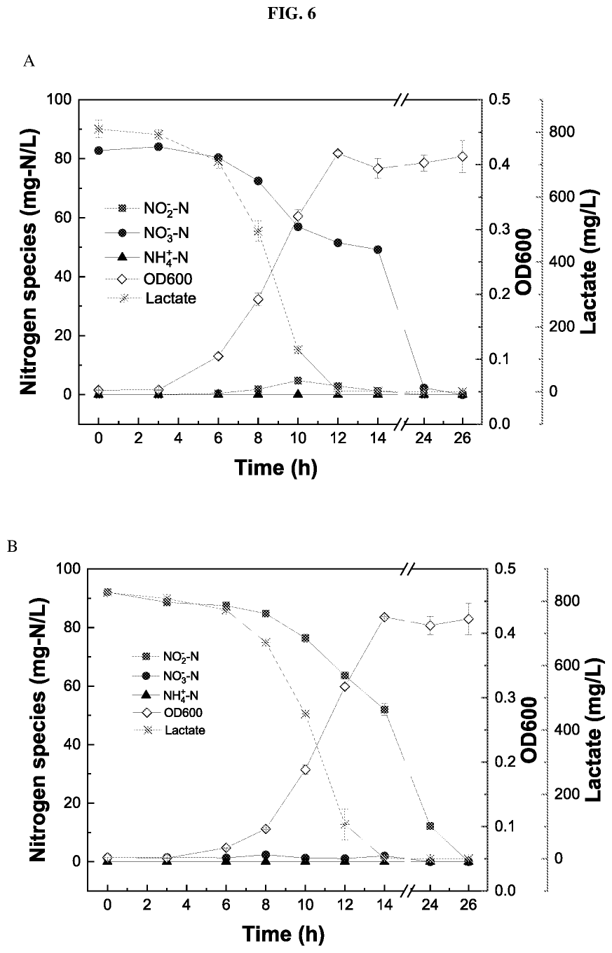

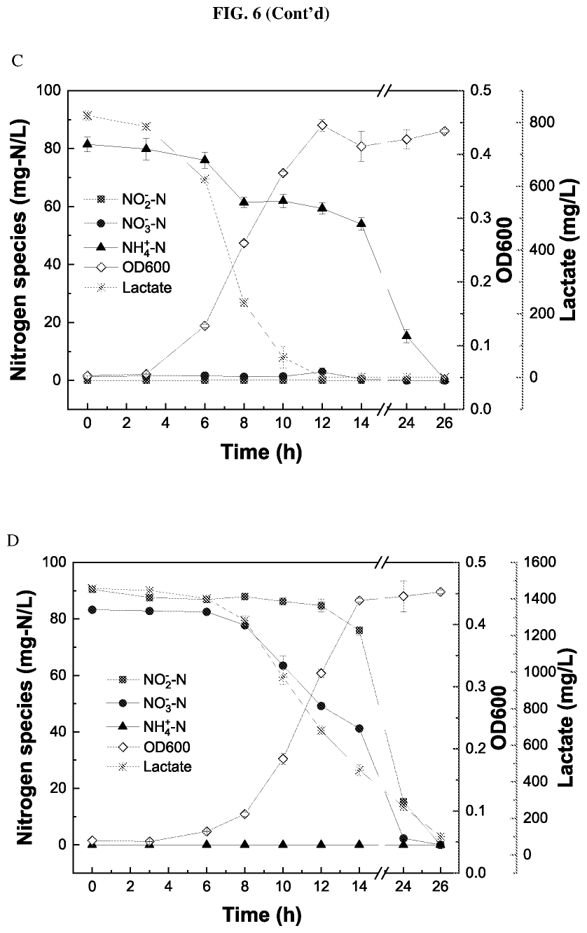

Denitrification by denitrifying bacteria typically proceeds as NO₃⁻→NO₂⁻→NO→N₂O→N₂, and is carried out by the nitrate reductase (narG), nitrite reductase (nirS), nitric oxide reductase (nor) and nitrous oxide reductase (nosZ) genes, respectively. Of these, only the nosZ gene was not detected in the genome of strain SND5 (FIG. 4). During denitrification of NO₃⁻ and NO₂⁻, strain SND5 produced N₂O without NO₂⁻ accumulation or changes in the N₂ concentration (FIG. 3). All these may suggest that N₂O is the end product of denitrification. The production of N₂O from both NO₃⁻ and NO₂⁻ without N₂ production suggests that denitrification in strain SND5 proceeds via NO₃⁻→NO₂⁻→NO→N₂O. The conventional ammonium oxidation pathway, NH₄⁺→NH₂OH→NO→NO₂⁻, is mediated by ammonia monooxygenase (AMO), encoded by the amo gene, and hydroxylamine oxidoreduc-tase (HAO), encoded by the hao gene. Neither of these genes was detected in the genome of strain SND5 (FIG. 4), which may suggest that ammonium oxidation in strain SND5 is distinct from this conventional pathway. To determine if heterotrophic ammonium oxidation by strain SND5 occurred via hydroxylamine (NH₂OH) as an obligate intermediate, NH₂OH was amended as the sole nitrogen source in a closed system. An increase in OD600 from 0.049±0.003 to 0.092±0.002 with concurrent decreases of hydroxylamine and lactate were observed (FIG. 5), indicating that strain SND5 could grow with hydroxylamine as the sole nitrogen source. This suggests that NH₂OH could be produced as an intermediate during heterotrophic ammonium oxidation, though it was not detected during our ammonium oxidation experiments. The removal rate of ammonium in the closed system was comparable to that in the open system (1.86 and 1.96 mg-N/L/h, respectively; FIG. 5a and FIG. 6c), suggesting that the effect on the removal rate of ammonium in a closed system was negligible. Because heterotrophic nitrification is a co-metabolic process, the removal of ammonium in the presence of oxygen depends on organic carbon in strain SND5. N₂O was not detected during either ammonium or hydroxylamine oxidation (FIGS. 5a and b). Interestingly, N₂ increased along with the decrease in O₂ during oxidation of ammonium and hydroxylamine (FIGS. 5a and b), which strongly suggest that N₂ was produced from this process. Rittmann and Mccarty have developed inorganic half-reactions indicating thermodynamic favorability of ammonium oxidation to nitrogen gas using oxygen as an electron acceptor (ΔG⁰=−1265.04 KJ/mol) without producing nitrous oxide as an intermediate. Indeed, this phenomenon has been previously reported, who observed N₂O and N₂ production in Alcaligenes faecalis strain NR during heterotrophic nitrification. Unlike strain SND5, however, N₂O was produced as a necessary intermediate in A. faecalis strain NR culture. A novel amo gene which is highly likely responsible for the above novel pathway—heterotrophic ammonium oxidation, was identified in the genome of strain SND5. As compared to the conventional amo gene, they have only ˜86% similarity making SND5's amo gene unique. Together, heterotrophic ammonium oxidation in strain SND5 should proceed via NH₄⁺→NH₂OH→N₂. The heterotrophic ammonium oxidation to nitrogen gas without emission of N₂O by strain SND5 is a particular advantage from the perspective of greenhouse gas emissions in WWTPs. As a greenhouse gas, N₂O shows 298 times the atmospheric heat-trapping capacity of CO₂, and its emission from WWTPs is associated with denitrification of nitrate and nitrite during biological nutrients removal. Since the majority of NO₂⁻ and NO₃⁻ in WWTPs is derived from ammonium oxidation, bypassing this pathway could have a major impact on overall N₂O production. Since strain SND5 generates neither NO₂⁻ or N₂O during heterotrophic ammonium oxidation, its metabolic activity could help to limit greenhouse emissions from WWTPs.

### Example 5

### Nitrogen Removal via Simultaneous Nitrification and Denitrification

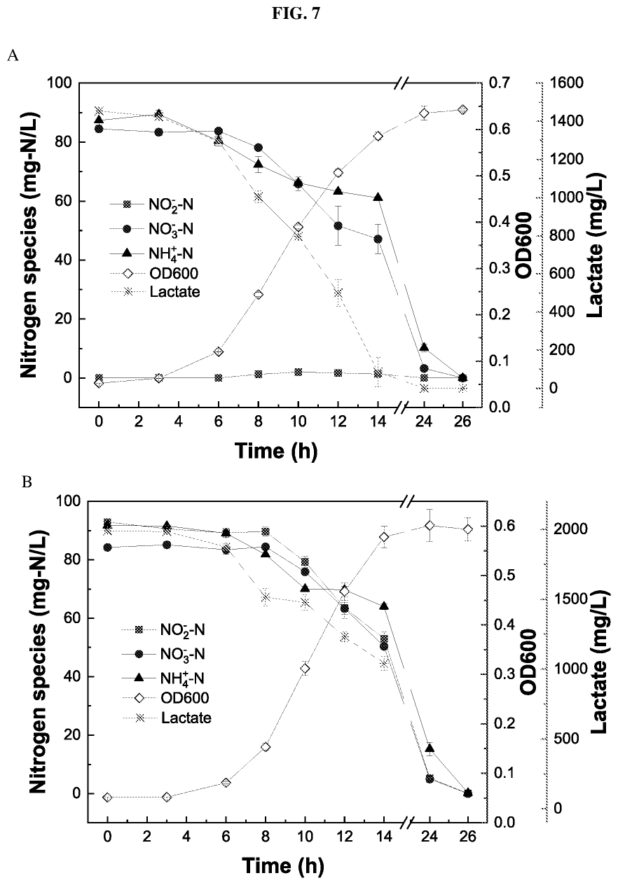

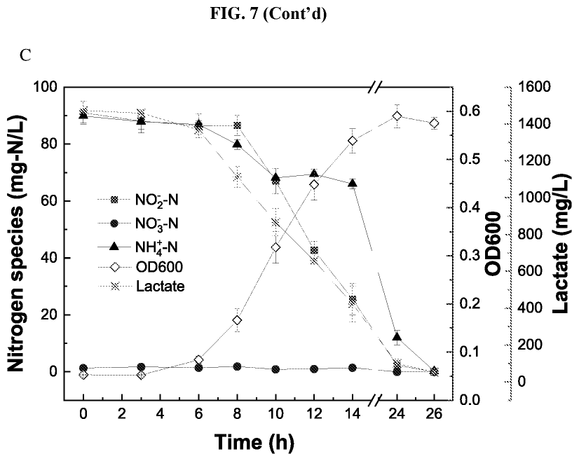

To investigate the versatility of culture SND5 on nitrogen removal under aerobic and heterotrophic conditions, the rates and completeness of aerobic denitrification of nitrate and nitrite, heterotrophic nitrification of ammonium, and simultaneous nitrification denitrification (SND) by culture SND5 were compared in batch tests (FIG. 7). The highest SND-mediated nitrogen removal efficiency was observed during logarithmic growth, and complete nitrogen removal occurred (100% nitrogen removal efficiencies) after approximately 26 h in all SND tests (FIG. 7). Consistent with abovementioned results, nitrite was not produced as an intermediate during either SND or acrobic denitrification with nitrate as the sole nitrogen source (FIG. 7a), suggesting that culture SND5 could denitrify nitrite immediately after generating it from nitrate. The rates of nitrate and ammonium removal (2.66 and 1.88 mg-N/L/h, respectively) by SND (FIG. 7a) were comparable to the rate of nitrate removal by aerobic denitrification and ammonium removal by heterotrophic nitrification (2.39 and 1.96 mg-N/L/h, respectively). Similar SND nitrogen removal rates were observed in cultures fed with nitrate, nitrite and ammonium (FIG. 7b), which were removed at 2.42, 2.85, and 1.98 mg-N/L/h, respectively. However, the rate of nitrite removal (4.68 mg-N/L/h) via SND supported by nitrite and ammonium as nitrogen sources (FIG. 7c) was higher than the rate of nitrite removal by both aerobic denitrification proceeding with nitrite as sole nitrogen source (2.86 mg-N/L/h) and with nitrite and nitrate as nitrogen sources (1.04 mg-N/L/h), suggesting that ammonium may somehow increase the rate of nitrite removal and that nitrate may slow it.

DO concentration higher than 0.5 mg/L but lower than 1.0 mg/L is suitable to sustain SND in wastewater treatment. Only with the first handed information of the DO effect on the SND, optimizations to wastewater treatment can be more efficient. The DO concentration variations were recorded during the SND tests by DK5 (Table 3). Before the SND can be proceeded by culture SND5 (before 6 hours), the DO values were above 3.00 mg/L in all tests including in the abiotic control, indicating that the oxygen was yet to be consumed during the lag phase. Later on, the DO values declined to around 1.00 mg/L while it was above 3.00 mg/L in the abiotic control, indicating the consumption of oxygen by culture SND5 during SND. Oxygen in the air can be re-dissolved in the medium after it was consumed, which explained why the DO values remained at around 1.00 mg/L during SND. These results may indicate that the optimal DO concentration for culture SND5 conducting SND would be around 1.00 mg/L. It was found that aerobic SND contributes to enhanced nitrogen removal under conditions of uneven or low DO concentrations. culture SND5 capable of conducting SND with DO concentration of around 1 mg/L provides insight to the application of SND in wastewater treatment.

TABLE 3
DO concentration during SND at different time points

| | DO concentration (mg/L) | | | | | | |
|---|---|---|---|---|---|---|---|
| Amendments | 0 h | 3 h | 6 h | 8 h | 10 h | 12 h | 14 h |
| NO₃⁻ & NH₄⁺ | 3.31 | 4.42 | 3.13 | 1.67 | 0.84 | 1.23 | 1.07 |
| NO₂⁻ & NH₄⁺ | 3.99 | 4.67 | 2.21 | 1.29 | 0.65 | 0.82 | 1.26 |
| NO₃⁻, NO₂⁻ & NH₄⁺ | 3.56 | 5.27 | 1.99 | 1.09 | 0.76 | 0.67 | 1.19 |
| Blank | 4.05 | 3.95 | 3.35 | 3.99 | 3.77 | 3.56 | 3.19 |

SND is an attractive strategy for treating nitrogen polluted wastewater because WWTPs with SND reaction do not require an anoxic tank, reducing the footprint of the plant. Even when growth medium contained multiple nitrogen sources, culture SND5 was able to denitrify nitrate and nitrite, and to nitrify ammonium simultaneously without nitrite accumulation under aerobic conditions (FIG. 7b). Previous investigations of SND in WWTPs have mainly focused on Pseudumonas and Bacillus for SND even though Thauera are commonly found in WWTPs. Our results suggest these previous analyses may have underestimated the contributions of Thauera to SND in WWTPs. The rapid denitrification and SND without nitrite accumulation exhibited by culture SND5 are potentially valuable metabolic properties in wastewater treatment operations seeking to remove different nitrogen species via SND.

### Example 6

### Culture SND5 Capable of Denitrifying Phosphate Accumulating

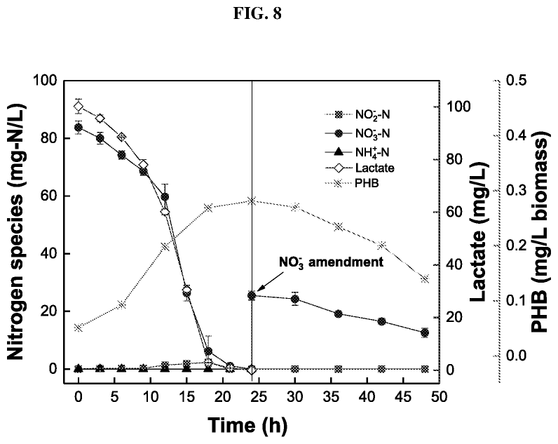

DPAOs are a metabolically interesting subgroup of denitrifying bacteria that produce and store polyhydroxybutyrate (PHB) during catabolismof extracellular carbon sources. The stored intracellular PHB can then be used by DPAOs to sustain the cell when external carbon sources are not available. Batch tests showed that culture SND5 accumulated PHB during lactate catabolismand subsequently utilized the intracellular PHB as a carbon source for nitrate denitrification when external carbon was depleted (FIG. 8). Phosphate accumulation in DPAOs relies upon phosphorylation and dephosphorylation of long-chain polyphosphates by polyphosphate kinase 1, which is encoded by the ppk1 gene. PCR using ppk1-specific primers revealed the presence of ppk1 in the genome of culture SND5, suggesting that culture SND5 might act as a DPAO.

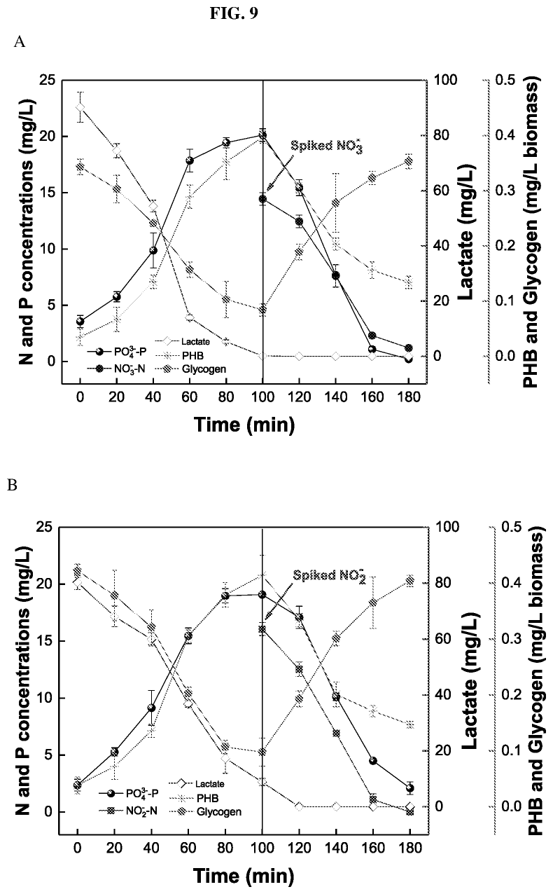

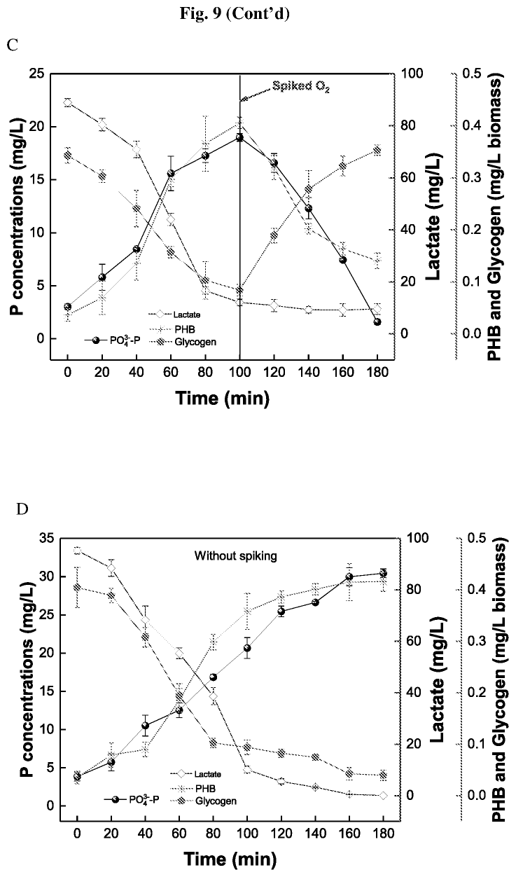

Further assays were performed to confirm uptake of extracellular phosphate during PHB catabolismby culture SND5 (FIG. 9). Intracellular PHB concentrations increased during lactate catabolismunder anaerobic conditions from ˜0.04 mg/L-biomass to a peaked amount of ˜0.40 mg/L-biomass; contemporaneous decreases in intracellular glycogen (from 0.35 mg/L-biomass to 0.10 mg/L-biomass) and increases in extracellular phosphate concentrations (from ˜2.00 mg/L to 20.00 mg/L) were also observed. Phosphate uptake was observed upon addition of nitrate, nitrite or oxygen (FIG. 9) with the phosphate concentration decreased by ˜21 mg/L, suggesting that these three substrates can all be used as electron acceptors in phosphate accumulation by culture SND5. No phosphate accumulation was observed without addition of an electron acceptor (FIG. 9d). Members of the candidate genus Accumulibacter are thought to be the main functional group responsible for phosphorus removal in wastewater, but observations of phosphate uptake by culture SND5 in this study suggest that the role of Ca. Accumulibacter in nutrients removal in WWTPs may have been overestimated. Together, these results indicate that culture SND5 is a nitrite-and nitrate-utilizing DPAO.

Members of the genus Thauera have previously been found to produce and consume polyhydroxylalkanoates (PHAs) to support metabolic nitrate and oxygen reduction, and to use a variety of external carbon sources to drive PHA synthesis. However, the uptake of phosphate during catabolismof an internally stored carbon source that we observed in culture SND5 has not been previously reported in Thauera. To the best of our knowledge, this is the first report of an SND bacterium that also functions as a DPAO. This seemingly unique phenotype of culture SND5 could be advantageous for nutrients removal in wastewater treatment systems, as competition for carbon sources between heterotrophs is a primary cause of underperformance in biological nutrient removal systems. Culture SND5, which can nitrify ammonium and denitrify nitrate/nitrite via SND and also remove phosphate when adequate carbon sources are available, could effectively outcompete less metabolically flexible heterotrophs and provide more reliable reactor performance.

### Example 7

### Reactor Development

Culture SND5 can be used to augment bioreactors treating nutrient-rich wastewater to introduce more reliable, low-cost and faster biological nutrients removal process. We have constructed a lab-scale bioreactor to develop a defined SND5-containing seed culture to enable more accurate prediction and assessment of reactor performance as well as to reduce start-up time for full-scale reactors.

A lab-scale (3.6 L) sequencing batch reactor was inoculated using cultures of strain SND5 and operated at a constant temperature of 30° C. The reactor used sequential anoxic and aerobic conditions in a 24 h cycle that was varied during development (Table 4). After each idle phase, 50- or 100-mL mixed liquor was discarded, and domestic wastewater was supplemented (50 or 100 mL) to keep the solids retention time (SRT) constant at 14 or 28 d, respectively; DO, ORP, and pH were monitored during operation.

TABLE 4
Operational parameters during reactor development

| | | | | Medium | | | | |
|---|---|---|---|---|---|---|---|---|
| | | | | introduction/ | | | TN | TP |
| | Anoxic | Aerobic | Settling | withdrawal | Idle | C/N | Removal | Removal |
| Phase | (h) | (h) | (h) | (h) | (h) | ratio | (%) | (%) |
| I | 22 | 0 | 1 | 0.5 | 0.5 | 2 | 66.4 ± 3.4 | n.a. |
| II | 19 | 3 | 1 | 0.5 | 0.5 | 2 | 77.2 ± 5.3 | 17.2 ± 7.0 |
| III | 18 | 4 | 1 | 0.5 | 0.5 | 2 | 86.1 ± 3.3 | 36.3 ± 10.8 |
| IV | 16 | 6 | 1 | 0.5 | 0.5 | 2 | 90.6 ± 1.2 | 59.4 ± 4.9 |
| V | 16 | 6 | 1 | 0.5 | 0.5 | 1 | 86.8 ± 0.9 | 51.3 ± 7.3 |

* Medium introduction phase is also known as filling phase, which lasts for 15 minutes. Medium withdrawal phase is also known as decanting phase, which lasts for 15 minutes.

Mixed liquor samples were drawn from the reactor at the start and end of each cycle for microbial community analysis and N and P profiling. Strain SND5 quickly became predominant in the reactor within 3 days, ultimately accounting for >90% of the microbial community. Interestingly, residual NO₃⁻ (8.4±2.0 mg-N/L) in the anoxic cycle was depleted in the aerobic cycle, possibly as part of polyhydroxybutyrate or trace organic carbon catabolismhe efficiency of N and P removal increased as aeration time was increased from 0 to 6 h in phases I-IV (C/N=2), with the highest degree of N and P removal occurring during phase IV. High nutrients removal efficiencies (N, 88.4±3.2%; P, 50.3±2.2%) were observed when SRT was increased from 14 to 28 d using the empirically determined optimal aeration (6 h, DO 0.8˜1 ppm) and C/N (C/N=2).

TABLE 5
Other sequences identified in strain SND5

| Sequence Description | SEQ ID NO: |
|---|---|
| Partial DNA sequence of 16S rRNA gene of strain SND5 | 17 |
| Full DNA sequence of 16S rRNA gene of strain SND5 | 18 |
| DNA sequence of amo gene of strain SND5 | 19 |
| DNA sequence of ppk1 gene of strain SND5 | 20 |
| DNA sequence of ppk2 gene of strain SND5 | 21 |

### Deposition of Microorganism

Depository authority: China General Microbiological Culture Collection Center (CGMCC)

Accession Number: CGMCC 21549

Deposition date: 28 Dec. 2020

### Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A method for water treatment comprising a step of contacting the water with a Thauera species that is capable of removing nitrogen via simultaneous nitrification and denitrification (SND) in the presence of a carbon source.

2. The method of claim 1, wherein nitrogen is removed without accumulation of nitrite (NO₂⁻) as an intermediate.

3. The method of claim 1, wherein nitrogen is removed without production of nitrous oxide (N₂O) as an end product, and the nitrogen source comprises ammonium (NH₄⁺).

4. The method of claim 1, wherein nitrogen is removed via heterotrophic ammonium oxidation proceeding as NH₄⁺→NH₂OH→N₂ or wherein nitrogen is removed via denitrification, and the nitrogen source comprises nitrate (NO₃⁻), nitrite (NO₂⁻), or a combination thereof.

5. (canceled)

6. The method of claim 4, wherein nitrogen is removed without the requirement of additional carbon sources after depletion of the carbon source or wherein the nitrogen removal efficiency is not compromised by high or low carbon:nitrogen (C/N) ratio.

7. (canceled)

8. The method of claim 7, wherein the C/N ratio is between about 1 and about 5, about 5, about 7.5, about 10, about 15, between about 8 and about 10, or between about 12 and about 32.

9. The method of claim 1, wherein nitrogen is substantially removed.

10. The method of claim 1, wherein ammonium (NH₄⁺), nitrate (NO₃⁻) and nitrite (NO₂⁻) are simultaneously removed at a rate of at least about 2.30, about 1.80 and about 2.00 mg-N/L/h, respectively.

11. The method of claim 1, wherein the Thauera species is further capable of removing phosphorous.

12. The method of claim 11, wherein phosphorous is removed via phosphate accumulating, and the phosphorous source in the water comprises phosphate (PO₄³⁻), hydrogen phosphate (HPO₄³⁻), dihydrogen phosphate (H2PO₄³⁻), or a combination thereof.

13. The method of claim 11, wherein phosphorous is removed without the requirement of additional carbon sources after depletion of the carbon source; optionally wherein phosphorous is removed at an efficiency of at least about 60%.

14. (canceled)

15. The method of claim 1, wherein the method is carried out under microoxic condition.

16. The method of claim 1, wherein nitrogen and phosphorous are removed via a single bioprocess.

17. The method of claim 1, wherein the carbon source comprises lactate, acetate, succinate, or a combination thereof.

18. The method of claim 1, wherein the Thauera species expresses one or more ppk genes selected from ppk1 gene, ppk2 gene, or a combination thereof; optionally wherein the ppk1 gene comprises the DNA sequence as set forth in SEQ ID NO: 20, and the ppk2 gene comprises the DNA sequence as set forth in SEQ ID NO: 21.

19. (canceled)

20. The method of claim 1, wherein the Thauera species expresses an amo gene encoding an ammonia monooxygenase that catalyzes the heterotrophic ammonium oxidation proceeding as NH₄⁺→NH₂OH→N₂.

21. The method of claim 20, wherein the amo gene comprises the DNA sequence as set forth in SEQ ID NO: 19.

22. The method of claim 1, wherein the Thauera species comprises a partial DNA sequence of 16S rRNA gene as set forth in SEQ ID NO: 17; optionally wherein the Thauera species comprises a full DNA sequence of 16S rRNA gene as set forth in SEQ ID NO: 18.

23. The method of claim 22, wherein the Thauera species is Thauera sp. strain SND5 (accession number: CGMCC 21549).

24. An isolated Thauera sp. strain SND5 (accession number: CGMCC 21549).

25. (canceled)

26. (canceled)

27. (canceled)

28. (canceled)

29. (canceled)

30. (canceled)
